(19) United States
(12) Patent Application Publication
(10) Pub. No.: US 2014/0106469 A1
(43) Pub. Date: Apr. 17, 2014

(54) Nanoparticle Plasmonic Sensor for Localized Surface Plasmon Resonance

(71) Applicant: Lawrence Berkeley National Laboratory - TTIPM, Berkeley, CA (US)

(72) Inventors: Hung-Jen Wu, College Station, TX (US); John T. Groves, Berkelely, CA (US)

(73) Assignee: THE REGENTS OF THE UNIVERSITY OF CALIFORNIA, Oakland, CA (US)

(21) Appl. No.: 14/052,652

(22) Filed: Oct. 11, 2013

Related U.S. Application Data
(60) Provisional application No. 61/712,749, filed on Oct. 11, 2012.

(51) Int. Cl. G01N 33/543

(52) U.S. Cl. CPC G01N 33/54346
USPC 436/501

(57) ABSTRACT

The present invention provides a sensor for detecting the binding of molecules to membrane surfaces. The sensor comprises a nanoparticle coated with a continuous layer of silica, and having a ligand attached thereto, for detection of an analyte in a solution. The nanoparticle can be further coated with a continuous membrane, such as a lipid bilayer.

### CROSS-REFERENCE TO RELATED APPLICATIONS

This application is a non-provisional patent application of and claims priority to U.S. Provisional Patent Application No. 61/712,749, filed on Oct. 11, 2012, which is hereby incorporated by reference in its entirety.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention relates to the fields of surface plasmonic sensing compositions, methods and devices for the detection of molecular binding on membrane surfaces.

### BACKGROUND OF THE INVENTION

The intracellular environment is dominated by membrane surfaces, and a significant fraction of biochemical processes involves membranes¹. Analytical methods for membrane analysis based on chemical labeling have many drawbacks, and hence there is substantial demand for quantitative label-free detection. Techniques, such as backscattering interferometry², colloidal assembly³, nanowire arrays⁴, microcantilevers⁵, acoustic sensing⁶, and surface Plasmon resonance⁷ have all been reported, but most are impractical for widespread adoption in biological laboratories. More promising for protein-lipid interactions is localized surface plasmon resonances (LSPR), in which binding causes measurable changes in refractive index⁸⁻¹¹. However, conventional LSPR techniques typically rely on analyte capture onto nanofabricated surfaces and often necessitate sophisticated instrumentation. The need for quantitative label-free detection methods that are simple, robustly reproducible, and accessible to scientists using generic laboratory equipment remains unmet.

### SUMMARY OF THE INVENTION

The present invention provides a sensor for detecting the binding of molecules to membrane surfaces. In some embodiments, the sensor comprises a polyhedral nanoparticle having a core coated with a continuous layer of silica (SiO₂), and further having a ligand attached to the nanoparticle. In some embodiments, the sensor comprises a nanoparticle comprising a core coated with a continuous layer of silica (SiO₂), and further coated with a continuous membrane, such as a lipid bilayer. In some embodiments, the sensor comprises silica-coated nanoscale silver cubes embedded in a phospholipid bilayer membrane that coats the entire surface of the silica-coated nanoscale silver cubes. In some embodiments, the silica-coated nanoscale silver cubes are on the surface of a substrate, such as a glass substrate, wherein the phospholipid bilayer membrane covers the entire substrate and the cubes. In some embodiments, the nanoparticles are in a solution.

In another aspect, the present invention provides a composition comprising, a nanoparticle having a continuous membrane coating optionally in contact with, or connected or attached to, a substrate. In some embodiments, the substrate is planar, spherical or a wall of a microfluidic channel. The membrane coating over the nanoparticle is a lipid bilayer or a hybrid lipid bilayer. In one embodiment, the nanoparticles comprise nanopolyhedras. In some embodiments, the nanopolyhedra is a nanocube. The nanoparticle core can comprise a metal, a semiconductor material, multi-layers of metals, a metal oxide, an alloy, a polymer, or carbon nanomaterials. In one embodiment, the nanoparticle core comprises metal, such as gold or silver. In some embodiments, the composition comprises a solution wherein the nanoparticle is in the solution.

In a further aspect, to form the hybrid lipid bilayer, the nanoparticles are chemically modified to display a self-assembled monolayer. In one embodiment, the membrane coating further comprises a ligand within the membrane. In another embodiment, the sensor further comprises a ligand capable of binding an analyte in a solution, wherein the ligand is a target molecule such as a protein, cell-surface protein, antibody, nucleic acid or a functionalized lipid headgroup or other biomolecule.

One aspect of the invention is a nano-plasmonic sensing device having simplicity of fabrication and of readout. In one embodiment, the readout is using simple absorbance spectrophotometry in an off-the-shelf instrument. The device presented herein is potentially easily parallelized for high-throughput applications, which distinguishes it from conventional SPR and related nanomaterial-based sensors.

The present invention provides for a sensor device comprising the composition of the present invention, including the nanoparticles of the present invention.

Thus the invention also provides a method comprising: (a) providing a solution, wherein the solution is suspected of containing a target molecule, (b) contacting the solution with a ligand conjugated to a nanoparticle of the invention and allowing the ligand conjugated to the nanoparticle to bind any target molecule present in the solution, and (c) detecting plasmon generated phenomena at the nanoparticle by the binding of the target molecule to the ligand conjugated to the nanoparticle.

In one embodiment, the plasmon-generated phenomena is optically detectable. In another embodiment, the step of detecting plasmon-generated phenomena comprises detecting light selected from absorbed light, reflected light, scattered light, or any combination thereof, and further wherein the method of detection comprises any combination selected from imaging, spectral characterization, intensity measurement, interferometry, and interference fringe analysis.

In another embodiment, the method further comprises: detecting a spectral shift in the known spectra of the nanoparticle, wherein such a spectral shift indicates the presence of the molecule possibly capable of binding the target molecule.

In one embodiment, the target molecule is a cell-membrane protein or a functionalized lipid headgroup.

In one embodiment, the sensor comprises a substrate having nanoparticles embedded on said substrate and a continuous supported lipid membrane coating said substrate and nanoparticles, wherein the nanoparticles are chemically modified to display a self-assembled monolayer such that subsequent exposure of the surface to lipid vesicles results in formation of a continuous lipid membrane coating the nanoparticles and the supporting substrate.

In another embodiment, a method for detecting an analyte of interest comprising the steps of: (a) providing a nanoparticle of the present invention, wherein the nanoparticle has a known spectra, and wherein the nanoparticle displays a ligand for the analyte of interest; (b) applying a sample suspected of containing a target analyte of interest to the nanoparticle; (c) detecting plasmon generated phenomena at the nanoparticle, whereby a spectral shift in the known spectra of the nanoparticle indicates that the target analyte is bound to the ligand.

The ligand can be oligonucleotides, ribonucleic acid residues, deoxyribonucleic acid residues, polypeptides, proteins, receptors, carbohydrates, a lipid-linked small molecule, thyroxine binding globulin, antibodies, enzymes, Fab fragments, lectins, nucleic acids, nucleic acid aptamers, avidin, protein A, barsar, complement component C1q, or other organic or inorganic molecules having a binding affinity for an analyte of interest.

Analytes or target molecules of interest that can be detected include nucleic acid molecules, proteins, peptides, haptens, metal ions, drugs, metabolites, pesticides, pollutants, toxins, hormones, enzymes, lectins, proteins, signaling molecules, inorganic or organic molecules, antibodies, contaminants, viruses, bacteria, other pathogenic organisms, idiotopes and cell surface markers.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. The physical properties of Ag@SiO₂ core-shell nanocube. (a) & (b) TEM images of Ag@SiO₂ nanocube. (a) is the close-up image of figure (b). (c)˜(f) The elemental maps obtained by high-angle annular dark field scanning TEM (HAADF-STEM) with energy dispersive x-ray spectroscopy (EDS). (c) to (f) represent silver, silicon, oxygen, and carbon, respectively. (g) Top: Detection procedure of nanocube sensors. Supported lipid bilayers are formed by vesicle fusion onto the silica surface, and protein binding is monitored by shifts in the LSPR extinction spectrum. Bottom: Typical spectra of membrane coverage and protein binding to the membrane surfaces. Sequential addition of lipid vesicles, BSA, and streptavidin causes LSPR red shifts. (h) Electric field norm (|E|/E₀) in decibel (dB) of a nanocube at resonance (n=1.33303, λ₀=474 nm) computed using finite-element analysis.

FIG. 2. Calibration of the nanocube assay. (a) Relation between LSPR shift and number of streptavidin per nanocube (left vertical axis) and surface density (right axis) measured by titration of biotinyl-cap-PE, titration of streptavidin, and fluorescence measurement of streptavidin concentration. Linear fit slopes are reported in Table 1. (b) Top: Concentrations of bound and unbound CTB are detected by multi-component FCS. Alexa 594-CTB binds to vesicles (average diameter 120 nm) containing 0.5% GM1 and 0.5% BODIPY-FL-DHPE lipids. BODIPY-FL-DHPE was used to determine the average number of vesicles diffusing within the excitation spot. Bottom: Binding kinetics measured by multi-component FCS and nanocube assay. (Error bar of FCS, n=20, mean±s.d.) CTB surface density was respectively calculated from known vesicle size and LSPR response to protein mass change in streptavidin-biotin systems (0.191 ng mm⁻² nm⁻¹). (c) Binding kinetics of wild-type and R407S K411S mutant of GST-Ste5 PH to different membrane surfaces. Concentrations of GST-Ste5 PH=1.6 μM; GST-Ste5 PH mutant=1.6 μM) (d) Equilibrium binding curves of GST-SteS PH to bilayers Kd=0.49±0.33 μM (PI(4,5)P2 bilayer) and 1.6±0.45 μM (P1(4,5)P2-free bilayer) (n=3, mean±s.e.m.) Error limits of K_{d} are derived from the statistical error of curve fitting.

FIG. 3: Scanning electron microscope (SEM) image of silver nanocube. Highly monodisperse nanocubes were synthesized using the polyol method.

FIG. 4: Energy-dispersive X-ray spectroscopy (EDS) spectra of Ag@SiO₂ nanocube. (a) EDS spectrum on the center of Ag@SiO₂ nanocube. (b) EDS spectrum on the silica shell of Ag@SiO₂ nanocube.

FIG. 5: Dark field light scattering of nanocube in different refractive index (R.I.) media (water/glycerol solution). The spectra were detected with an inverted microscope coupled to a spectrometer. The inset shows the resolved details of quadrupolar peak. The dashed lines represent the position of maximum peak.

FIG. 6: The light scattering spectra detected at a fixed angle (90 degree) in different refractive index (R.I.) media (water/glycerol solution) in a standard fluorescence spectrophotometer. The inset shows the resolved details of the quadrupolar peak. The dashed lines represent the position of the maximum peak.

FIG. 7: Electromagnetic field enhancement profile along the nanocube diagonal computed in FEA. The cross-section originates from the nanocube's center through a corner along the vector (x,y,z)=(1,1,−1) in FIG. 1h. The model geometry of the silver nanocube was calculated from TEM, revealing a nanocube lateral dimension of 98 nm, 19 nm radius of curvature at the edges. The geometry of the silica shell was directly scaled up from silver nanocube to reach 4.0 nm shell thickness on facet, and thus the shell thickness through the corner is 7.0 nm in this cross-section.

FIG. 8: Normalized fluorescence recovery of supported lipid bilayers over three different substrates: (1) a bare glass surface, (2) Ag@SiO₂ nanocube adhered on a glass surface, and (3) Ag nanocube adhered on a glass surface. Nanocube-adhered substrates were prepared by drying a solution of nanocubes onto glass (2·10⁸ nanocubes on 18 mm circle microscope cover glass). The two surfaces are expected to have similar nanocube densities after immobilization. No difference in recovery was observed between glass and Ag@SiO₂ nanocube substrates, although a higher immobile fraction was observed on the Ag nanocube substrate. Illustrations are not drawn to scale.

FIG. 9: The kinetics of streptavidin binding to biotinylated lipid at different concentrations monitored by nanocube sensors. The biotinylated bilayer contains 3% biotinyl-cap-PE and 97% DOPC. The control bilayer is 100% DOPC. Fifteen consecutive LSPR spectra were collected to obtain an average baseline prior to kinetics measurements. Higher concentrations of streptavidin result in stronger shifts in the LSPR spectra. Streptavidin does not bind in the negative control bilayer (100% DOPC) and expectedly shows no LSPR shift.

FIG. 10: The LSPR shift of Ag and Ag@SiO₂ nanocubes in various refractive index media (water/glycerol solution). The averages and standard deviations of 3 different synthesis batches are presented. Ag@SiO₂ nanocubes show less sensitivity to refractive index change of media. The Ag nanocubes had a shift of 169 nm/RIU whereas the Ag@SiO₂ nanocubes had a shift of 123 nm/RIU.

FIG. 11: The correlation between maximum absorbance of quadrupolar peaks and nanocube concentration. Nanocubes deposited onto sedimentation chambers were directly imaged by dark field scattering microscopy. The linear relation between particle concentration and absorbance provides an approach to easily determine the nanocube concentration during the binding measurement¹. (n=20, mean±s.d.)

FIG. 12: Estimated error of LSPR measurement. (a) LSPR spectra with various nanocube concentrations. The symbols and solid lines represent the raw data and the polynomial fits at different nanocube concentrations (solid volume fraction 0 and maximum absorbance A). Lower concentrations of nanocubes show a lower signal-to-noise ratio and result in larger deviations of polynomial fits. (b) The standard error of 20 continuous measurements at different nanocube concentrations. (n=3, mean±s.d.)

FIG. 13: CTB binding measurements using FCS and nanocube assay. (a) The kinetics of Alexa 594-CTB binding to vesicles containing G_{M1}, lipid monitored by multi-component FCS. (n=20, mean±s.d.) (b) The kinetics of Alexa 594-CTB binding to supported lipid bilayer on Ag@SiO₂ nanocubes.

FIGS. 14 A, B, C and D in general illustrate the properties of the nanocube system. The graph in the lower left corner (B) shows the LSPR shift relative to the concentration of Antigen or Ligand as termed here. The detection limit is labeled as the smallest shift in LSPR signal that is detectable using this system. The general reaction mechanism is illustrated in the upper left panel (A) for a Ligand-Receptor equilibrium reaction such as between an antibody and antigen. The graphs in (C) of the properties of the nanocubes where each graph is labeled with properties such as the standard error associated with each measurement as a function of both the concentration of cubes in solution and the solid volume fraction, the electric field enhancement as a function of the distance from the surface of the cube, along with an SEM of the actual nanocubes to illustrate not only the actual shape but the monodispersity of the sample population. FIG. 14D provides further details of the nanocubes described.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to a “nanoparticle” includes a single nanoparticle, as well as a plurality of nanoparticles, either the same (e.g., the same shape) or different.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

In one embodiment, the present invention provides for a label-free optical detection tool capable of monitoring localized surface plasmon resonance (LSPR) in nanocubes, such as silver nanocubes. The platform can observe molecules, such as proteins and biomolecules, binding at lipid membrane surfaces in real-time. The optical sensors are nanoparticles, the nanoparticles having a core having a silica shell. In some embodiments, the nanoparticles are 100 nm silver nanocubes covered with an about 3.9 nm thick silica layer. Lipid bilayers self-assemble on the nanoparticles which create a continuous and fluid mimic of cell membrane with which to study biomolecular interactions. The sharp quadruple LSPR peak of a nanocube, such as a silver nanocube, is highly sensitive to the refractive index of its surrounding environment. Measurement of shifts in this resonance is the basis of detection, as molecules that bind or unbind to the lipid surface change the nanocube's effective refractive index. Extinction spectra can be easily monitored by transmission in an ultraviolet-visible (UV-vis) spectrophotometer or by scattering in a dark field microscope, both of which are common and general laboratory equipment. This method is capable of measuring protein binding kinetics and the specificity of biomolecular interactions.

The ability to mix and detect the analyte in membrane-based measurements is a unique capability of this sensor. The platform also enables existing assays, such as those designed for SPR, to be performed more accurately and more productively. The present invention comprises one or more, or all, of the following features which make the sensor superior to existing techniques:

(1) LSPR effects are highly localized, leading to high sensitivity and low background noise. At resonance, an amplified electromagnetic field penetrates about 10 nm into the medium from the silica surface, and is strongest at the nanocube's corners. This short skin depth means that only molecules very close to the sensor's functionalized surface contribute to its signal. This reduces the background noise from molecules in solution when compared with conventional SPR, which has a far longer (200 nm) field penetration depth.

(2) With nanocubes, the nanocube silica coating or the stable lipid bilayer is readily functionalized on the nanocube surface. The silver nanoparticle's cube shape provides a low-curvature surface, while the silica shell confers compatibility with lipid molecules. These conditions cause lipid bilayers to self-assemble. Previous LSPR biosensing studies, for example those utilizing spherical gold particles to detect antibody-antigen binding, attach biomolecules to a highly curved surface. For this reason, it is not possible to form stable membrane on other nanoparticles, with which no membrane binding experiments have been reported.

(3) Read-out of the LSPR signal is very simple. LSPR spectra can be acquired with well established optical techniques, such as transmission/absorption measurements in a UV-vis spectrometer. Beyond this common instrument, no special apparatus is required.

(4) Large quantities of the nanoparticles can be synthesized at low cost. Nanoparticles, such as the silica-coated silver nanocubes can be produced using a one-pot reaction. Established techniques such as SPR and nanowire-based assays require specialized, expensive nanofabrication processes. In contrast, the present technique can easily synthesize very large quantities of nanocube sensors with minimal preparation.

(5) Detection in the solution phase. Compared to most label-free detection techniques, measurements from the present nanocubes are conducted in the solution phase. One is able to simultaneously monitor a large quantity of sensors (over about 10¹² nanocubes) to obtain the ensemble average of binding events which can reduce the particle-to-particle variation in LSPR response. Solution-phase sensors can also easily be multiplexed or parallelized. For example, this can be achieved by performing binding measurements in 96-microwell plates and acquiring LSPR spectra in a dark field microscope or plate absorbance reader. Additionally, solution-phase measurements can be easily performed in standard microfluidic devices or a commercial micro-volume spectrometer, such as Nanodrop. This can minimize the usage of protein, lipids, and other expensive reagents.

In an embodiment where the nanoparticle is coated with a lipid bilayer, one or more of the following technical issues are overcome: poor self-assembly of lipid bilayers on nanoparticles, the contribution of background signal to surface measurements, and aggregation of particles once they are functionalized. To address the first issue, a thin uniform layer of silica is coated on the nanocube. Secondly, the unique shape of the nanocube localizes the electromagnetic field near the particle's surface. This enhanced field penetrates into and about 10 nm past the silica layer, focusing the range of detection so that the sensor is much more sensitive to molecules at its surface than those from the surrounding solution. Thirdly, the stable fluid bilayer passivates the silica surface and prevents aggregation of nanocube sensors.

The present invention provides a sensor for detecting the binding of molecules to a ligand attached to either the nanoparticle surface or the lipid bilayer/membrane surface. In one embodiment, the sensor comprises a nanoparticle having a continuous membrane coating, and optionally the nanoparticle is disposed on a substrate. The nano-plasmonic sensing device is intended to have simplicity of fabrication and of readout. In one embodiment, the manufacture of the basic sensor surface is based on a series of solution-based deposition and wash steps, and the readout is using simple absorbance spectrophotometry in an off-the-shelf instrument. The sensor presented herein is potentially easily parallelized for high-throughput applications, which distinguishes it from conventional SPR and related nanomaterial-based sensors.

In one embodiment, a multiplexable, label-free sensor device to measure interfacial binding of an analyte at a phospholipid membrane surface. In one embodiment, the sensor device comprises a nanoparticle embedded in a lipid bilayer, and the lipid bilayer coated nanoparticle optionally displayed on a surface of a substrate. The lipids themselves, or biomolecules embedded into the bilayers, of the nanoparticle determine the analyte specificity of the device. Binding occurs either to the membrane directly, or to biomolecules including but limited to antibodies, small molecules, proteins or nucleic acids, or membrane-associated proteins, lipids or elements.

In another embodiment, a multiplexable, label-free sensor device to measure interfacial binding of an analyte to a ligand. In one embodiment, the sensor device comprises a nanoparticle having a ligand attached on the surface of the nanoparticle, thereby providing a ligand-nanoparticle conjugate. In some embodiments, the ligand-nanoparticle conjugate is suspended in solution-phase or displayed on a surface of a substrate and allowed to contact a solution suspected of having an analyte in the solution. The lipids themselves, or biomolecules embedded into the bilayers, of the nanoparticle determine the analyte specificity of the device. Binding occurs between the ligand and the analyte to be detected in a solution. The ligand may be biomolecules including but not limited to antibodies, small molecules, proteins, nucleic acids, membrane-associated proteins, lipids or elements. Suspected analytes may be

In some embodiments, a device and assay and methods are described which can measure binding by exploiting the optical absorbance due to localized surface plasmon resonance (LSPR) scattering by the nanoparticles. A polyhedral nanoparticle shape such as a nanocube provides the LSPR scattering spectrum of sharply defined peaks, the positions of which are dependent on the refractive index of the surrounding environment, and hence to analyte bound to the ligand on the nanoparticle or the membrane bilayer surrounding the nanoparticle. Spectral shifts of the peaks indicate binding or unbinding of the analyte to ligand and/or the bilayer surface. In one embodiment, the device is easily realized, for example, as a simple flow chamber that may be placed in an absorbance spectrophotometer, where the nanoparticle scattering registers as a distinct absorbance spectrum. This device is capable of collecting binding kinetics data as well as specificity measurements, all without depending on potentially disruptive analyte labeling. In another embodiment, an assay is carried out by providing the ligand-nanoparticle conjugate to a solution and suspending the nanoparticles in the solution to allow binding to any analyte in the solution. The binding event is detected using for example, an absorbance spectrophotometer.

The present embodiment lacks the burdensome technical requirements of other devices, such as micropatterning of substrates. In contrast, nanocubes can be synthesized en masse and easily deposited over large areas. This means that this system is potentially easily multiplexed/parallelized and automated. For example, this could be achieved by using our basic technique adapted to a glass-bottomed 96-well plate and read in a plate reader absorbance spectrophotometer. Thus, in another embodiment, the present device provides for methods for detecting an analyte of interest or assays for biodetection.

An instrumental development that enables certain embodiments is the capability of producing defect-free, fluid lipid bilayers that coat the nanoparticles. Bilayers will form on the silica of the nanoparticle under a specific range of conditions. The bilayer preserves the environmental sensitivity of the nanoparticles' spectrum, and also allows the LSPR spectrum to be easily interrogated. The quadripolar LSPR peak allows the accurate determination of the peak maximum beyond the resolution limit of the spectrophotometer. This is necessary for monitoring small shifts in the nanoparticle spectrum. In some embodiments, a small, continuous-flow chamber is used to contain the nanoparticles to enable fluid exchange over the membrane surface during data collection, though not all applications may require it.

In some embodiments, the substrate comprises materials such as glass, mica, quartz, polydimethylsiloxane (PDMS), polystyrene, silica, SiO₂, MgF₂, CaF₂, polyacrylamide, and various polysaccharides including dextran, agarose, cellulose and modified, crosslinked and derivatized embodiments thereof, and any other materials with constant spectra or any lipid-compatible material, i.e., a bilayer will form on the surface. For example, polymers like PDMS, or substrates like glass that have been decorated with biomolecules which can support lipid membranes (e.g. polymer supported bilayers) {See Tanaka, M.; Sackmann, E. Nature 2005, 437, 656-663, Sackmann, E. Science 1996, 271, 43-48} and can be suitable substrates. SiO₂ is a particularly effective substrate material, and is readily available in the form of glass, quartz, fused silica, or oxidized silicon wafers. These surfaces can be readily created on a variety of substrates, and patterned using a wide range of micro- and nano-fabrication processes including: photolithography, micro-contact printing, electron beam lithography, scanning probe lithography and traditional material deposition and etching techniques.

In another embodiment, the nanoparticles are other polyhedra including but not limited to, nanocubes, nanopyramids, nanobowties, nanorods, nanocrescents, nanotubes, nanowontons, nanodisks, layered nanodisks with an alternating shielding layer, and other nanoscale polyhedra.

The nanoparticle core can comprise a metal, a semiconductor material, multi-layers of metals, a metal oxide, an alloy, a polymer, or carbon nanomaterials. In certain embodiments the nanoparticle core comprises a metal selected from the group consisting of Ga, Au, Ag, Cu, AI, Ta, Ti, Ru, Ir, Pt, Pd, Os, Mn, Hf, Zr, V, Nb, La, Y, Gd, Sr, Ba, Cs, Cr, Co, Ni, Zn, Ga, In, Cd, Rh, Re, W, Mo, and oxides, and/or alloys, and/or mixtures, and/or nitrides, and/or sintered matrix thereof.

In one embodiment the nanoparticles are silver or gold nanocubes. The remarkably sharp quadripolar resonance peak of silver nanocubes allows us to resolve more subtle variations in the spectrum compared with the very broad scattering signatures of other nanoparticles.

In one embodiment, the nanoparticles can be made according to the methods described in A. Tao, P. Sinsermsuksakul, and P. Yang. Tunable plasmonic lattices of silver nanocrystals. Nature Nanotechnology, 2(7):435-440, July 2007 and A. Tao, P. Sinsermsuksakul, and P. D. Yang. Polyhedral silver nanocrystals with distinct scattering signatures. Angewandte Chemie-International Edition, 45(28):4597-4601, 2006, both of which are hereby incorporated by reference.

The nanoparticle core is coated with a silica or other biocompatible shell. In some embodiments, the silica shell thickness is optimized to about 3.9 nm, which provides a sufficiently thin shell such that the binding event is within the 10-20 nm detection range, where the 10-20 nm is measured as the distance from the core nanoparticle surface to the outer edge of the shell. This presents a biocompatible surface upon which lipids self-assemble, resulting in a stable lipid bilayer which coats the surface of the nanoparticle.

In some embodiments, the polyhedral nanoparticle has an edge length of about 100 nm to 500 nm. In more preferred embodiments, the polyhedral nanoparticle has an edge length of about 100 nm. Having a larger edge length enables ease in nanoparticle fabrication and washing steps, and enables for bench-top centrifugation and washing and does not require ultra-centrifugation. However too large of a size affects the localized SPR of the particle and monodisperity of the particles.

Co-pending U.S. patent application Ser. No. 13/204,506, filed on Aug. 5, 2011, entitled, “Plasmonic System for Detecting Binding of Biological Molecules,” hereby incorporated by reference in its entirety, discloses a sensor comprising nanoscale silver cubes deposited on a glass surface and which are embedded in a phospholipid membrane that coats the entire surface of the device. Co-pending U.S. patent application Ser. No. 12/151,553, filed on Jul. 21, 2008, entitled, “A Fluid Membrane-Based Ligand Display System for Live Cell Assays and Disease Diagnosis Applications,” hereby incorporated by reference in its entirety, discloses detection of cell phenotypes in a soluble lipid bilayer (SLB) assay using soluble signaling ligands attached to the lipid bilayers. Other SLB assays are described in U.S. Pat. No. 6,228,326, which is incorporated by reference in its entirety. Co-pending U.S. patent application Ser. No. 10/076,727, incorporated by reference in its entirety, describes use of SLB assays to effect and modulate cell adhesion. All these related publications and patent applications are incorporated by reference in their entirety, especially for the purposes of enabling and exemplifying aspects of the present invention that had been developed in previous work conducted by some of the same inventors.

In some embodiments, the supported bilayer of the assay system comprises a lipid bilayer wherein the primary ingredient is an egg-phosphatidylcholine (PC) membrane. In the absence of dopants, cells do not adhere to this membrane. Other suitable lipids that do not permit cell adhesion include pure phosphatidylcholine membranes such as dimyrstoyl-phosphatidylcholine or dipalmitoylphosphatidylcholine. Another suitable primary lipid component is phosphatidylethanolamine (PE), which is also, in addition to PC, a primary component.

The lipid composition in the supported lipid bilayer can comprise dopants to vary bilayer properties. Particular dopant lipids are a negatively, positively or neutrally charged lipid. In one embodiment, the dopant lipid is the negatively charged lipid phosphatidylserine (PS). Other potential dopants can be dipalmitoylphosphatidic acid (PA), distearoylphosphatidylglycerol (PG), phosphatidylinositol, 1,2-dioleoyl-3-dimethylammonium-propane, 1,2 dioleoyl-3-trimethylammonium-propane (DAP), dimethyldioctadecylammonium bromide (DDAB), 1,2-dioleoyl-sn-glycero-3-ethylphosphocholine (ethyl-PC), N-(7-nitrobenz-2-oxa-1,3-diazol-4-yl)-1,2-dihexadecanoyl-sn-glycero-3-phosphoethanolamine ammonium salt (NDB-PE). Suitable neutral lipid dopants include cerebrosides and ceramides. The amount of the dopant is selected based on the property of the dopant. For a lipid dopant, 2 to 10%, up to 20% is preferred.

In contrast to other metal nanoparticle-based systems, the data collection technique described measures a signal derived from large populations of nanoparticles, which means particle-to-particle variation in LSPR response is averaged. This helps ensure the comparability of one device to another. Thus, in some embodiments, there is a population density of ˜10-100 particles/μm² density on a surface.

To facilitate simple fabrication of the sensors, a method for fabrication was designed. In one embodiment, the manufacture of the basic sensor surface is based on a series of solution-based deposition and wash steps, and the readout is using simple absorbance spectrophotometry in an off-the-shelf instrument.

In some embodiments, the bilayer-coated nanoparticles are coated with a polymer such as polyvinylpyrrolidone and its derivatives. The polymer-coated nanoparticles are dried on a substrate, such as a surface, such as planar surface, of a substrate, in a solvent.

In some embodiments, the planar surface is a glass slide, a microfluidic device, or glass surface having a flow chamber to allow the sample suspected of containing an analyte to interact with the membrane-coated device. In other embodiments, rather than the flow chamber, the surface of a glass-bottomed multi-well plate could be used, and thus allowing the assay to be multiplexed and enabling a readout in a plate reader or spectrophotometer.

The nanoparticles may be adsorbed onto other surfaces instead of a substantially planar surface. In one embodiment, the surface is a bead similar to that in copending U.S. patent application Ser. No. 10/581,371, the contents of which are herein incorporated by reference. Specific examples of the particles include polystyrene, cellulose, dextran crosslinked with bisacrylamide (Biogel™, Bio-Rad, U.S.A.), agar, glass beads and latex beads. The beads may be nanometer to micrometer scale in diameter. This would enable LSPR readout of surfaces from suspension rather than on a monolithic surface (e.g., in a cuvette).

In some embodiments, the ligand is attached to the nanoparticle. In some embodiments, the ligand is attached to the nanoparticle shell by means of a linker. The linker can include functional groups such as a silane, thiol or epoxy group. Examples of such linkers and reactions to link ligands to the shell are known in the art, and described in for example, US 2011-0046018 A1, hereby incorporated by reference. The Examples below also describe methods and examples of linking agents to link ligands to the nanoparticle.

The term “analyte”, “analyte of interest”, or “target analyte” or “target molecule” or “target biomolecule” refers to the compound or composition to be detected, including drugs, metabolites, pesticides, pollutants, and the like. The analyte can be comprised of a member of a specific binding pair (sbp) and may be a ligand, which is monovalent (monoepitopic) or polyvalent (polyepitopic), preferably antigenic or haptenic, and is a single compound or plurality of compounds, which share at least one common epitopic or determinant site. The analyte can be a part of a cell such as bacteria or a cell bearing a blood group antigen such as A, B, D, etc., or an HLA antigen or a microorganism, e.g., bacterium, fungus, protozoan, or virus. If the analyte is monoepitopic, the analyte can be further modified, e.g. chemically, to provide one or more additional binding sites. In practicing this invention, the analyte has at least two binding sites.

The term “ligand” refers to any organic compound for which a receptor naturally exists or can be prepared. The term ligand also includes ligand analogs, which are modified ligands, usually an organic radical or analyte analog, usually of a molecular weight greater than 100, which can compete with the analogous ligand for a receptor, the modification providing means to join the ligand analog to another molecule. The ligand analog will usually differ from the ligand by more than replacement of a hydrogen with a bond, which links the ligand analog to a hub or label, but need not. The ligand analog can bind to the receptor in a manner similar to the ligand. The analog could be, for example, an antibody directed against the idiotype of an antibody to the ligand.

The term “receptor” or “antiligand” refers to any compound or composition capable of recognizing a particular spatial and polar organization of a molecule, e.g., epitopic or determinant site. Illustrative receptors include naturally occurring receptors, e.g., thyroxine binding globulin, antibodies, enzymes, Fab fragments, lectins, nucleic acids, nucleic acid aptamers, avidin, protein A, barsar, complement component C1q, and the like. Avidin is intended to include egg white avidin and biotin binding proteins from other sources, such as streptavidin.

The ligand may be an oligonucleotide of ribonucleic acid residues, deoxyribonucleic acid residues, polypeptides, proteins, receptors, carbohydrates, thyroxine binding globulin, antibodies, enzymes, Fab fragments, lectins, nucleic acids, nucleic acid aptamers, avidin, protein A, barsar, complement component C1q, organic or inorganic molecules having a binding affinity for an analyte of interest, or lipid-linked small molecules that are displayed, bound or otherwise attached to the membrane coating the sensor.

The term “specific binding pair (sbp) member” refers to one of two different molecules, which specifically binds to and can be defined as complementary with a particular spatial and/or polar organization of the other molecule. The members of the specific binding pair can be referred to as ligand and receptor (antiligand). These will usually be members of an immunological pair such as antigen-antibody, although other specific binding pairs such as biotin-avidin, enzyme-substrate, enzyme-antagonist, enzyme-agonist, drug-target molecule, hormones-hormone receptors, nucleic acid duplexes, IgG-protein A/protein G, polynucleotide pairs such as DNA-DNA, DNA-RNA, protein-DNA, lipid-DNA, lipid-protein, polysaccharide-lipid, protein-polysaccharide, nucleic acid aptamers and associated target ligands (e.g., small organic compounds, nucleic acids, proteins, peptides, viruses, cells, etc.), and the like are not immunological pairs but are included in the invention and the definition of sbp member. A member of a specific binding pair can be the entire molecule, or only a portion of the molecule so long as the member specifically binds to the binding site on the target analyte to form a specific binding pair.

The term “specific binding” refers to the specific recognition of one of two different molecules for the other compared to substantially less recognition of other molecules. Generally, the molecules have areas on their surfaces or in cavities giving rise to specific recognition between the two molecules. Exemplary of specific binding are antibody-antigen interactions, enzyme-substrate interactions, polynucleotide interactions, and so forth.

The analyte of interest may be nucleic acid molecules, proteins, peptides, haptens, metal ions, drugs, metabolites, pesticide or pollutant. The method can be used to detect the presence of such analytes as toxins, hormones, enzymes, lectins, proteins, signaling molecules, inorganic or organic molecules, antibodies, contaminants, viruses, bacteria, other pathogenic organisms, idiotopes or other cell surface markers. It is intended that the present method can be used to detect the presence or absence of an analyte of interest in a sample suspected of containing the analyte of interest.

In some embodiments, the target analyte is comprised of a nucleic acid and the specific binding complement is an oligonucleotide. Alternatively, the target analyte is a protein or hapten and the specific binding complement is an antibody comprising a monoclonal or polyclonal antibody. Alternatively, the target analyte is a sequence from a genomic DNA sample and the specific binding complement are oligonucleotides, the oligonucleotides having a sequence that is complementary to at least a portion of the genomic sequence. The genomic DNA may be eukaryotic, bacterial, fungal or viral DNA.

In one embodiment, detection of a particular cytokine can be used for diagnosis of cancer. Specific analytes of interest include cytokines, such as IL-2 as shown in the examples. Cytokines are important analytes of interest in that cytokines play a central role in the regulation of hematopoiesis; mediating the differentiation, migration, activation and proliferation of phenotypically diverse cells. Improved detection limits of cytokines will allow for earlier and more accurate diagnosis and treatments of cancers and immunodeficiency-related diseases and lead to an increased understanding of cytokine-related diseases and biology, because cytokines are signature biomarkers when humans are infected by foreign antigens.

Chemokines are another important class of analytes of interest. Chemokines are released from a wide variety of cells in response to bacterial infection, viruses and agents that cause physical damage such as silica or the urate crystals. They function mainly as chemoattractants for leukocytes, recruiting monocytes, neutrophils and other effector cells from the blood to sites of infection or damage. They can be released by many different cell types and serve to guide cells involved in innate immunity and also the lymphocytes of the adaptive immune system. Thus, improved detection limits of chemokines will allow for earlier and more accurate diagnosis and treatments, i.e. for bacterial infections and viral infections.

In some embodiments, the target analyte may be a variety of pathogenic organisms including, but not limited to, sialic acid to detect HIV, Chlamydia, Neisseria meningitides, Streptococcus suis, Salmonella, mumps, newcastle, and various viruses, including reovirus, sendai virus, and myxovirus; and 9-OAC sialic acid to detect coronavirus, encephalomyelitis virus, and rotavirus; non-sialic acid glycoproteins to detect cytomegalovirus and measles virus; CD4, vasoactive intestinal peptide, and peptide T to detect HIV; epidermal growth factor to detect vaccinia; acetylcholine receptor to detect rabies; Cd3 complement receptor to detect Epstein-Ban virus; β-adrenergic receptor to detect reovirus; ICAM-1, N-CAM, and myelin-associated glycoprotein MAb to detect rhinovirus; polio virus receptor to detect polio virus; fibroblast growth factor receptor to detect herpes virus; oligomannose to detect Escherichia coli; ganglioside G_{M1} to detect Neisseria meningitides; and antibodies to detect a broad variety of pathogens (e.g., Neisseria gonorrhoeae, V. vulnificus, V. parahaemolyticus, V. cholerae, and V. alginolyticus).

In some embodiments, multiple analytes of interest can be detected by utilizing multiple ligands specific to different analytes of interest and utilizing distinct barcode oligonucleotides corresponding to each analyte of interest.

The analyte of interest may be found directly in a sample such as a body fluid from a host. The host may be a mammal, reptile, bird, amphibian, fish, or insect. In a particular embodiment, the host is a human. The body fluid can be, for example, urine, blood, plasma, serum, saliva, semen, stool, sputum, cerebral spinal fluid, tears, mucus, pus, phlegm, and the like. The particles can be mixed with live cells or samples containing live cells.

Where the sample is live cells or samples containing live cells, a cell surface protein or other molecule may serve as the analyte of interest. This allows for the detection of cell activation and proliferation events, cellular interactions, multiplexing, and other physiologically relevant events

The target molecule binding as well as target molecule adhesion to a cell can be detected by any method of detection including but not limited to detection by absorbed light, reflected light, scattered light, back reflected interference fringes, or scattered reflected intergerence fringes, light from resonant energy transfer energy of the plasmonic field coupled to fluorophores (like fluorescence resonance energy transfer).

In another embodiment, the sensor can be an array of individually addressable regions of substrate (e.g., wells in a microwell plate, or channels in a microfluidic chip) to form a multiplex assay that allows testing different events in different wells, or channels.

In one embodiment, absorbance or reflectance spectra of the entire substrate is measured. The image and spectrum of the sensor can be acquired using a dark-field microscopy system with a true-color imaging camera and a spectrometer. For example, the microscopy system can consist of a Carl Zeiss Axiovert 200 inverted microscope (Carl Zeiss, Germany) equipped with a darkfield condenser (1.2<NA<1.4), a true-color digital camera (CoolSNAP cf, Roper Scientific, NJ), and a 300 mm focal-length and 300 grooves/mm monochromator (Acton Research, MA) with a 1024×256-pixel cooled spectrograph CCD camera (Roper Scientific, NJ). After photobleaching the fluorescence, the true-color scattering images of the nanoparticles are taken using a 60× objective lens (NA=0.8) and the true-color camera with a white light illumination from a 100 W halogen lamp.

In another embodiment, rather than measuring the absorbance spectrum of the entire substrate, interrogation of individual nanoparticles or regions/clusters of nanoparticles is contemplated. Moreover the sensor could record scattered light instead of an absorbance spectrum. The scattering spectra of the nanoparticles can be taken using the same optics, but they are routed to the monochromator and spectrograph CCD. Furthermore, a 2 μm-wide aperture can be placed in front of the entrance slit of the monochromator to keep only a single nanoparticle in the region of interest.

Raw spectra are normalized with respect to the spectrum of a non-resonant nanoparticle (i.e., polystyrene) after the background subtraction. In the spectroscopy experiments, the nanoparticle-immobilized glass slide can be mounted on a transparent ITO heater with an external thermostat. The nanoparticles can be immobilized and immersed in a drop of buffer solution which also serves as the contact fluid for the dark-field condenser. The distance between the condenser and nanoparticles can be ˜1-2 mm. The sample suspected of containing an analyte to be detected can be loaded by pipette into the contact fluid and the continuous spectrum acquisition started simultaneously. The microscopy system can be completely covered by a dark shield, which prevents ambient light interference and serious evaporation of the sample.

In one embodiment, the analyte density is calculated by considering the fluorescence of the analyte bound to identical bilayers as herein described and in Galush et al. Biophys J, 2008, which is hereby incorporated by reference, and demonstrated by the Examples infra. Furthermore, other ways to calibrate the analyte density can be employed. For example, instead of fluorescence, one could use mass standards. In one instance, another protein binding in known amounts to the same or identical substrate can be calculated.

In another embodiment, sensor response could be measured by localizing the spectrum peak by position of maximum signal, position of centroid, or absolute intensity (spectrum height). The sensor response could be measured by monitoring the increase in fluorescence emission of the analyte upon binding to the membrane.

In yet another embodiment, darkfield microscopy of the whole substrate, portions of the substrate, or individual particles could be used as the readout.

In another embodiment, for real-time plasmon resonance sensing of molecular binding or interactions, the continuous acquisition of the scattering spectrum of a selected nanoparticle starts in synchronization with the introduction of the sample suspected of containing the analyte. For example, one spectrum is taken every minute with a 10-second integration time. The plasmon resonance wavelength data exhibits a first-order exponential decay. Calibration curves generated by plasmon resonance sensing of multiple analytes can be generated and typical scattering spectra and plasmon resonance peak wavelengths of the nanoparticle after the interactions and reactions with multiple analytes can be acquired. In one embodiment, the curve is fit from a semi-empirical model using a Langevin-type dependence of the refractive index vs. amount of unbound ligand or analyte.

And in another embodiment, surface enhanced Raman spectroscopy (SERS) can be used to perform the detection and the readout instead of absorbance (see McFarland: 2005, Porter: 2008). A typical SERS experimental system configuration comprising a microscopy system with Raman spectrometer used to acquire Raman scattering spectra from single tagged nanoplasmonic resonators. In a particular embodiment, the system is comprised of inverted microscope equipped with a digital camera and a monochromator with a spectrograph CCD camera, a laser source and an optical lens. In one embodiment, Raman spectra can be measured using a modified inverted microscope, such as the Carl Zeiss Axiovert 200 (Carl Zeiss, Germany), with a 50× objective in a backscattering configuration. The laser wavelength can be in the visible and near infrared region. In a particular embodiment, a 785 nm semiconductor laser is used as the excitation source of Raman scattering, and the laser beam is focused by a 40× objective lens on the NPR. The 785 nm or other near infrared light source can assure less absorption by the biological tissue and lower fluorescence background. However, for certain applications, lower wavelength excitation light might be more advantageous, and even UV light excitation can be used for applications. The excitation power can also be measured by a photometer to insure an output of ˜0.5 to 1.0 mW. The Raman scattering light is then collected through the same optical pathway through a long-pass filter and analyzed by the spectrometer. The Raman spectrometer can be linked to a computer whereby the spectrometer can be controlled and the spectra can be obtained and a spectrograph can be observed. The spectral detection can be done with ordinary spectral polychrometer and cooled CCD camera. In an embodiment where the ligands and analytes are nucleotides, the monitored wavenumbers of Raman peaks can range from 400 cm⁻¹ to 2000 cm⁻¹.

In one embodiment, the sensor is incubated with a sample suspected of containing the biomolecule to be detected, preferably in a closed transparent microchamber. The microchamber is mounted on a 37° C. thermal plate on an inverted Raman microscope with darkfield illumination for nanoparticle visualization. The nanoparticles are visualized using the darkfield illumination from oblique angles as the bright dots. The excitation laser is focused on the nanoparticles by a microscopy objective lens. A SERS signal is collected by the same objective lens and analyzed by a spectrometer.

In some embodiments, the sensor can be used to measure supported bilayer formation or change in supported bilayer physical properties, in aggregate or on a microscopic scale.

In another embodiment, the sensor can be used to quantify cell adhesion to the substrate mediated by a membrane-resident molecule. As cells tightly bind to the surface and closely adhere, this should change the LSPR scattering signature. In another embodiment, the sensor can be used to monitor lipid vesicle/micelle/bicelle binding.

In some embodiments, using a microscope, one can address different regions of the substrate independently. This could be on the single- or multi-nanoparticle scale. This could be done using darkfield microscopy, or localized illumination or scattering sensor to see the LSPR signature. Notably, SPR is not spatially resolved, whereas the present invention can be.

The present sensor is not bound by the described applications but is contemplated to find use in sensing and detection in various SPR methods and devices.

In some embodiments, the invention provides for kits for the practice of the methods described herein. In some embodiments, the kits provide the nanoparticles and reagents to coat the nanoparticles with the bilayer and/or ligand and instructions such as those provided in the Examples. In other embodiments, the kits may also provide collection and/or processing materials or devices to collect specimens to be tested, along with the test reagents or devices to measure LSPR for detection.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Membrane-Protein Binding Measured with Solution-Phase Plasmonic Nanocube Sensors

We describe a solution-phase sensor of lipid-protein binding based on localized surface plasmon resonance (LSPR) of silver nanocubes. When silica-coated nanocubes are mixed into a suspension of lipid vesicles, supported membranes spontaneously assemble on their surfaces. Using a standard laboratory spectrophotometer, we calibrate the LSPR peak shift due to protein binding to the membrane surface and then characterize the lipid-binding specificity of a pleckstrin-homology domain protein.

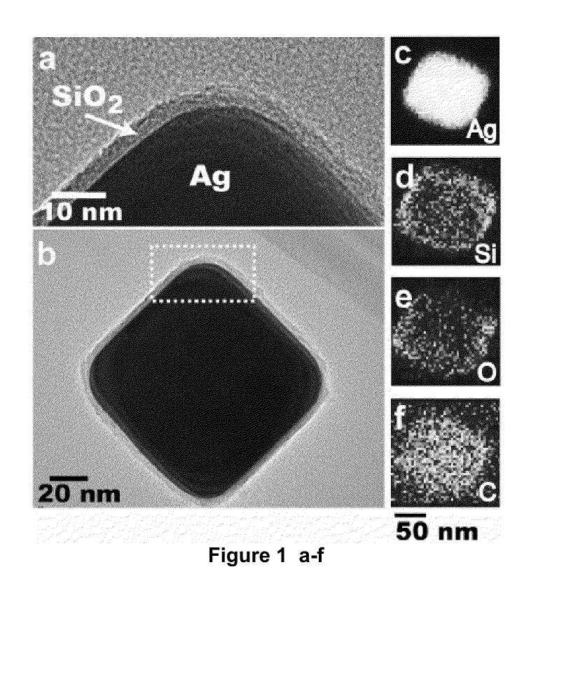

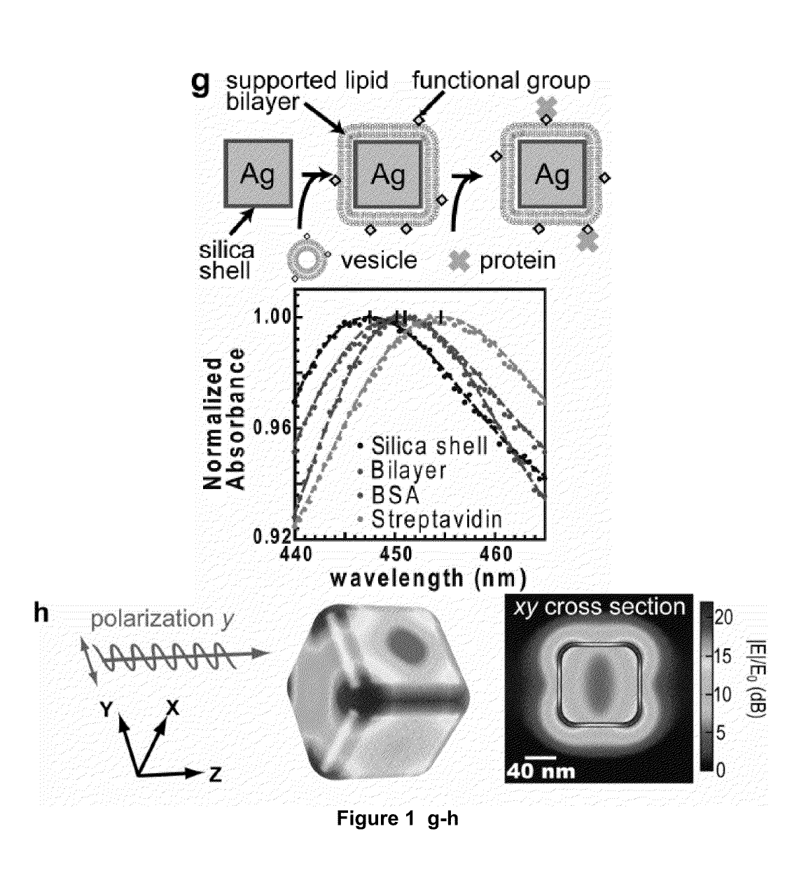

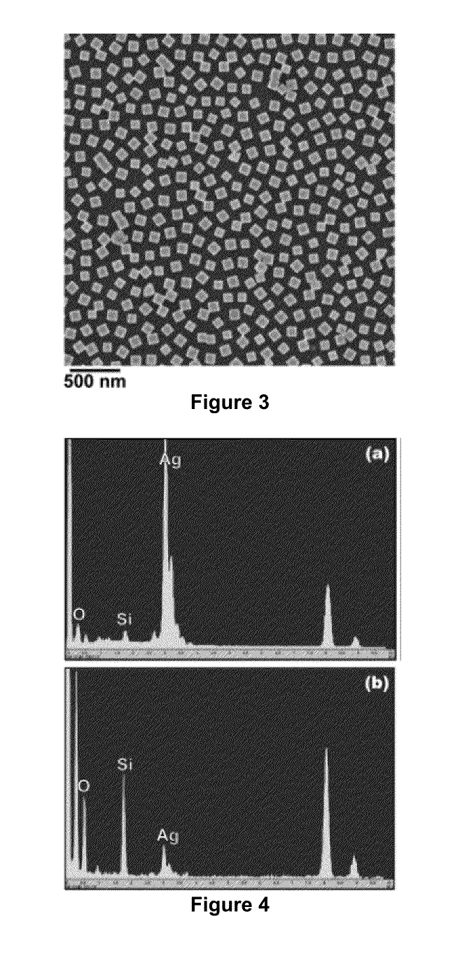

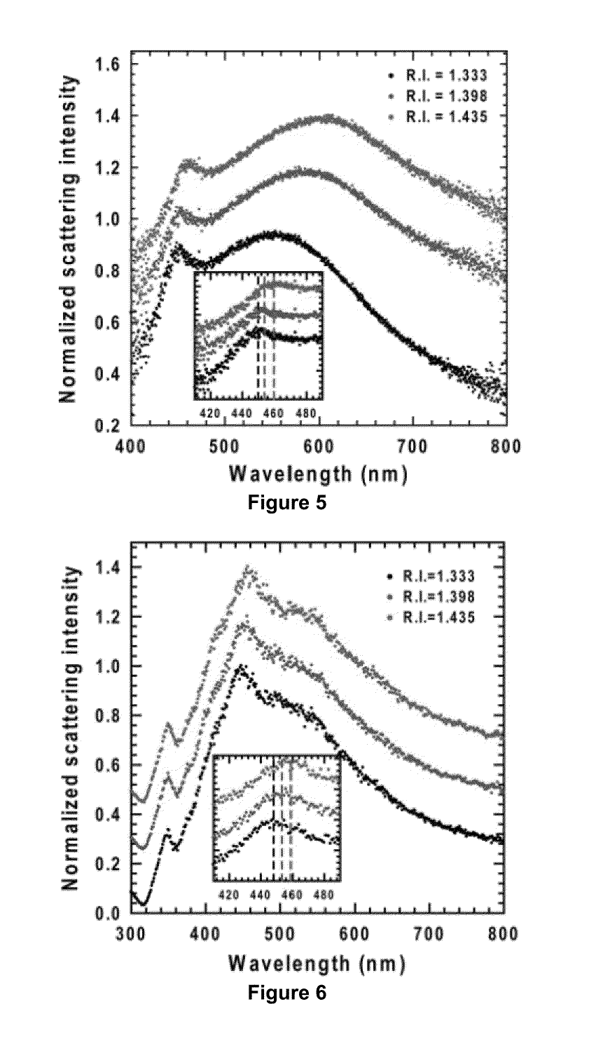

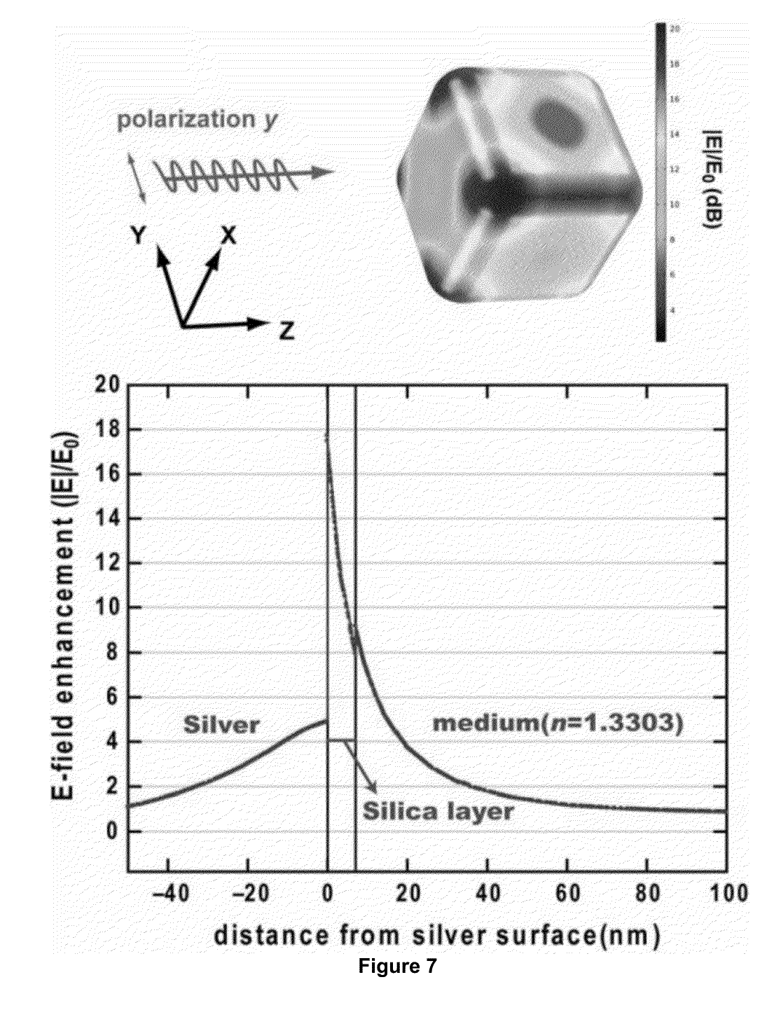

Here, we report a platform that enables label-free measurements of protein binding to membrane surfaces on a standard laboratory spectrophotometer. We have previously described label-free detection using the LSPR of thiolated silver nanocubes immobilization on flat substrates.⁹ This configuration required multiple reactions, a customized detection system, and ultimately proved similarly impractical as the other methods mentioned above. A substantial improvement in utility is achieved here by modifying the system to allow measurements to be performed entirely in the solution phase. Highly monodisperse Ag nanocubes were prepared by an established synthetic protocol^{l2} (FIG. 3). In order to create a favorable surface for membrane assembly and suspension in solution, an ultra-thin layer of silica was then grown using Stöber synthesis (Methods). Transmission electron microscopy (TEM) micrographs revealed a uniform silica shell covering the Ag surface with average thickness 3.9±0.2 nm (n=5, mean±s.d.) and corners with curvature radius of 19 nm (FIGS. 1a and 1b). Elemental maps acquired by high-angle annular dark field scanning TEM show that the silicon and oxygen intensities were strongest on the edges of Ag@SiO₂ core-shell nanocube particles (silver core @ silica shell), indicating the shell is conformal and uniform (FIG. 1c-1f, and FIG. 4). Additionally, the SiO₂ coating provides a shelf life in excess of one year by slowing silver oxidation. Ag@SiO₂ nanocubes exhibit a sharp quadrupolar LSPR scattering peak (FIG. 1g). This is easily observed in the extinction spectrum of a suspension of nanocubes using standard laboratory tools such as a transmission ultraviolet-visible (UV-vis) spectrophotometer, microvolume spectrometer (e.g. NanoDrop), dark-field microscopy (FIG. 5), or light scattering spectrophotometer (FIG. 6). Electromagnetic simulations based on the actual particle geometry confirm the time-averaged electric field norms exhibit quadrupole resonance with the highest near-field enhancement near the nanocube corners (FIG. 1h). At quadrupole resonance, |E|/E0 decays to 50% of its value at the silica-media interface over about 10 nm distance. The silica layer is sufficiently thin that the LSPR field still penetrates a lipid bilayer of 3-5 nm thickness (FIG. 7). A widely used figure of merit (FOM) for LSPR is the peak shift per refractive index unit (nm/RIU) normalized to the linewidth of the LSPR peak (details in Method section). The FOM for Ag@SiO₂ nanocubes is 1.7 versus 2.4 for bare silver nanocubes.

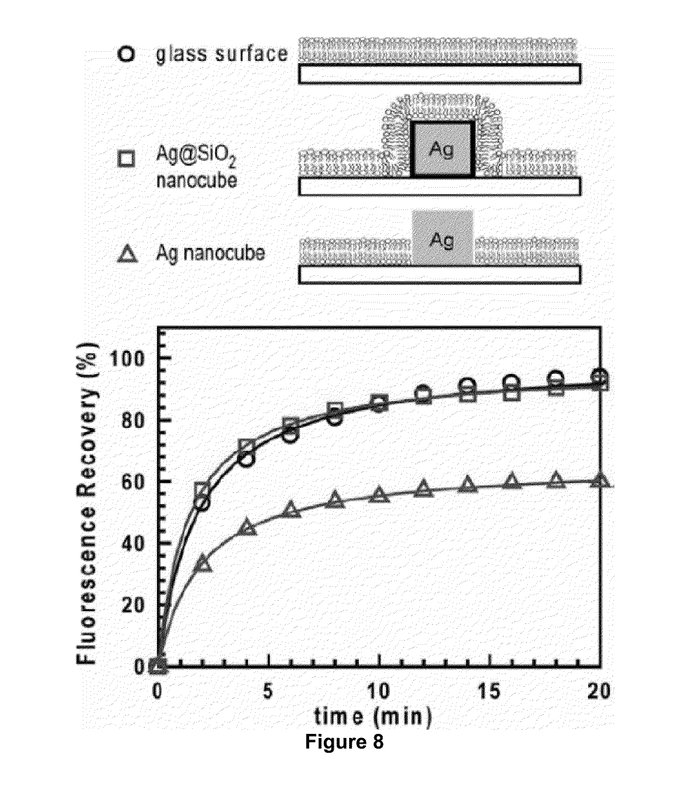

Supported lipid bilayers form spontaneously upon mixing Ag@SiO₂ nanocubes into a lipid vesicle suspension (FIG. 1g). Supported membrane formation was confirmed using fluorescence recovery after photobleaching (FRAP) experiments to test the lateral fluidity and connectivity of membranes covering substrate-adsorbed nanocubes⁹ (FIG. 8). The nanocubes were first immobilized on planar glass substrates and then exposed to lipid vesicle suspensions so that a supported lipid bilayer formed on top of both the glass substrate and nanocubes. Bilayers on Ag@SiO₂ nanocube-covered substrates exhibited almost identical recovery behavior to bilayers on bare glass (FIG. 8). This result indicates that the supported bilayers on Ag@SiO₂ nanocubes are fluid and connected to the bilayer on surrounding glass. The magnitude of fluorescence recovery also confirms that the majority of nanocubes are covered with lipid membrane⁹. In contrast, bilayers on a bare Ag nanocube covered substrates exhibited similar recovery times but only 60% of the recovery on bare glass, which illustrates that lipids absorbed on bare nanocubes did not form a fluid and continuous bilayer with the surrounding fluid bilayer. Although it has been suggested that supported lipid bilayer cannot form on a highly curved surfaces (11 nm radius of curvature) due to high elastic energy¹³, we did not observe any such limitation on the Ag@SiO₂ nanocubes (19 nm radius of curvature over corner).

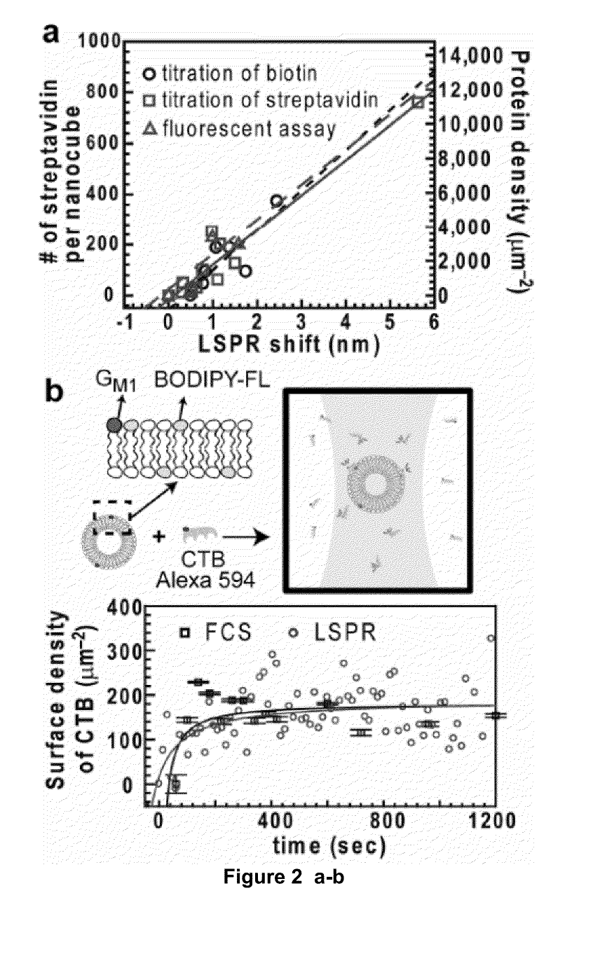

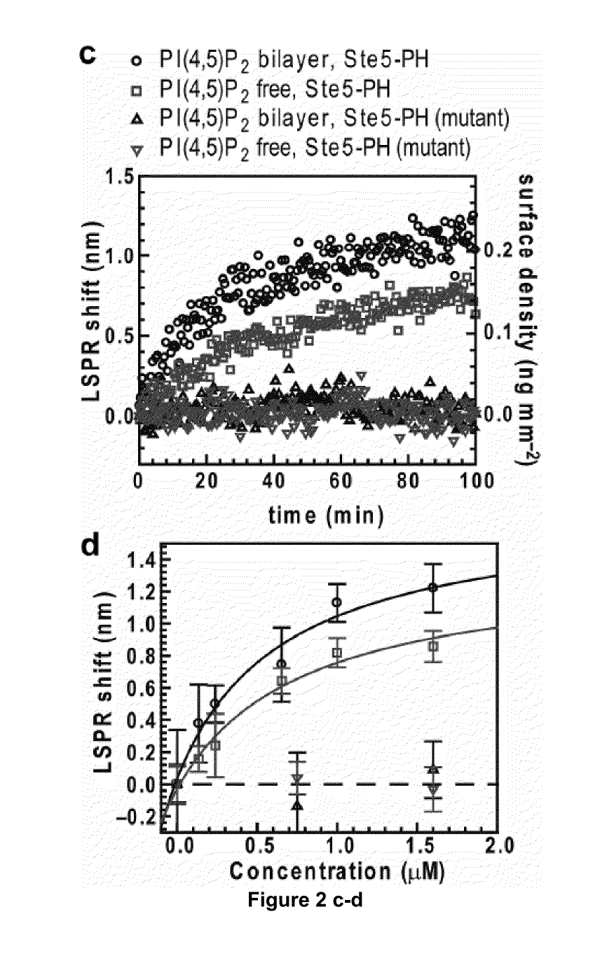

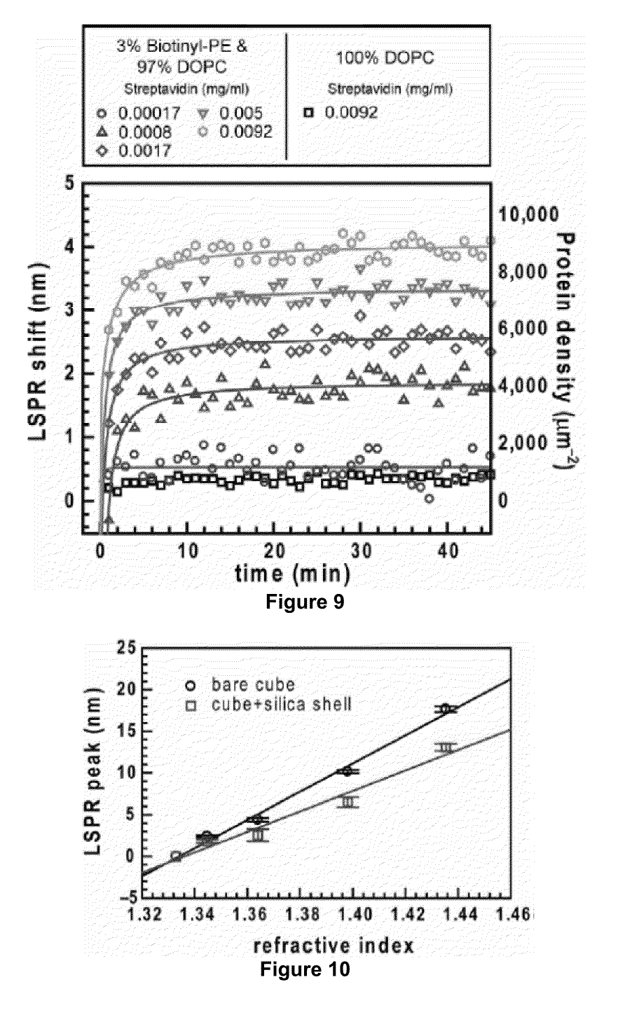

The LSPR response of the system is calibrated by monitoring the essentially irreversible binding of streptavidin to biotinylated lipids in the nanocube supported membrane (FIG. 9). We employed three different approaches to control the surface density of membrane-bound streptavidin: (i) titrating biotinyl-cap-PE in bilayer; (ii) titrating streptavidin in solution; and (iii) measuring unbound fluorescent streptavidin. LSPR shifts were measured at different known surface densities of streptavidin and exhibited a linear relation with protein density (FIG. 2a). Consistent LSPR responses of 0.191±0.025 ng mm-2 nm-1 (n=3, mean±s.d.) were determined by three independent approaches (Table 1).

TABLE 1
The summary of protein surface density per LSPR peak shift.
The protein densities per LSPR shift measured by streptavidin
titration, biotinyl-cap-PE titration, and fluorescence assay were
evaluated from the slopes in FIG. 2a. The value measured by
FCS is calculated from the average LSPR shift after 1000 sec
in FIG. 2b. The average response determined by biotin-streptavidin
system was 0.191 ng mm⁻² nm⁻¹, consistent with the FCS
measurements. Error limits are derived from the statistical error
of curve fitting.

| | | |
|---|---|---|
| # of protein/ | protein number | |
| nanocube/ | density/ | protein mass |
| LSPR | LSPR shift | density/LSPR shift |
| shift (nm⁻¹) | (μm⁻² nm⁻¹) | (ng mm⁻² nm⁻¹) |

Streptavidin-biotin system

| | | | |
|---|---|---|---|
| Streptavidin titration | 138 ± 12 | 2033 ± 180 | 0.178 ± 0.016 |
| biotin titration | 135 ± 37 | 1996 ± 57 | 0.175 ± 0.048 |
| fluorescence assay | 170 ± 23 | 2512 ± 352 | 0.220 ± 0.031 |
| | | Average: | 0.191 ± 0.025 |

CTB-G_{M1} system

| | | | |
|---|---|---|---|
| FCS | 141 ± 16 | 2084 ± 234 | 0.191 ± 0.021 |

To assess that bilayer-coated Ag@SiO₂ nanocubes can quantify protein binding accurately, we compared the system with the established method of multi-component fluorescence correlation spectroscopy (multi-component FCS)¹⁴. Cholera toxin subunit B (CTB) binding to the membrane-associated receptor GM1 was used as a model system (FIG. 2b). In multi-component FCS measurements, lipid vesicles and CTB were labeled with different fluorophores and the concentrations of bound and unbound CTB were monitored. The average size of vesicles was determined independently by dynamic light scattering, which allowed determination of the surface density of vesicle-bound CTB. Using the same materials and under the same experimental conditions, nanocube measurements were performed independently. LSPR response was converted to protein surface density using the LSPR response to protein mass change measured in the biotin-streptavidin system, 0.191 ng mm⁻² nm⁻¹ (Table 1). Kinetics measured by multi-component FCS and nanocube methods reached equilibrium state and the same surface density after 1000 sec (FIG. 2b). It is worth noting that unlike FCS, which only works at low concentration, the nanocube detection strategy has a much broader working range.

Finally, we used the Ag@SiO₂ nanocube assay to examine the heretofore unknown lipid binding specificity of a prototypic mitogen-activated protein kinase (MAPK) scaffold protein, Ste5. Ste5 contains a pleckstrin-homology domain (PH domain, residues 388-518) that is essential for its membrane recruitment and function, but the dependence of Ste5 binding on membrane composition is not well known¹⁵. We investigated the binding of Ste5 to membranes with and without PI(4,5)P2. GST-Ste5 PH domain fusion proteins (corresponding to Ste5 residue 369-517), with and without R407S and K411S mutations thought to abrogate lipid binding, were constructed, expressed, and purified from Entamoeba coli. To avoid interference of detergent with the membrane assay, we eliminated its use during protein purification. Only wildtype GST-Ste5 PH domain bound to the membrane surface (FIG. 2c). Although more Ste5 binding was observed on PI(4,5)P2 membranes, appreciable binding was also observed on membranes without PI(4,5)P₂. This may be due to the presence of phosphatidic acid lipids, which have been observed to association with PH domains in other protein systems¹⁶. Binding curves were established to compute the binding affinity of GST-Ste5 on different compositions of membranes (FIG. 2d). At similar lipid compositions, we have previously reported rough estimates of Kd for Ste5-membrane binding using filter-immobilized lipids, liposome flotation assays, and surface plasmon resonance (SPR), that suggest a dissociation constant in the 5-10 μM range¹⁵. However, the lipid immobilization and tethering required for the filter and SPR assays are strongly disruptive of the membrane surface environment⁷ and liposome flotation assays are intrinsically error-prone. Thus, among all of the measurements, we consider the nanocube assay to be the most consistent and most accurate.

We report a core-shell Ag@SiO₂ nanocube sensor that can measure protein binding to its membrane-coated surfaces. No complicated fabrication is necessary and these sensors can be prepared on the gram scale (>10¹⁴) at minimal cost. Solution phase measurements readily integrate 10¹² nanocubes in the illumination area of a standard spectrophotometer cuvette. This provides sensitivity of approximately 0.19 ng cm⁻² based on 0.01 nm standard error of 20 consecutive LSPR measurements, in contrast to the immobilized format⁹ (10⁹ nanocubes; sensitivity=1.5 ng cm²). This method is applicable to analytes that bind lipid membranes or membrane proteins, including proteins, peptides, nucleic acids, or even entire cells. The biggest advantage of this method is that simply adding Ag@SiO₂ nanocubes to a vesicle suspension produces a system in which analytes binding to the membrane surface can be read out by standard spectral technique widely available in most labs, without labeling.

### Calibration of Nanocube Concentration and Error of LSPR Measurement

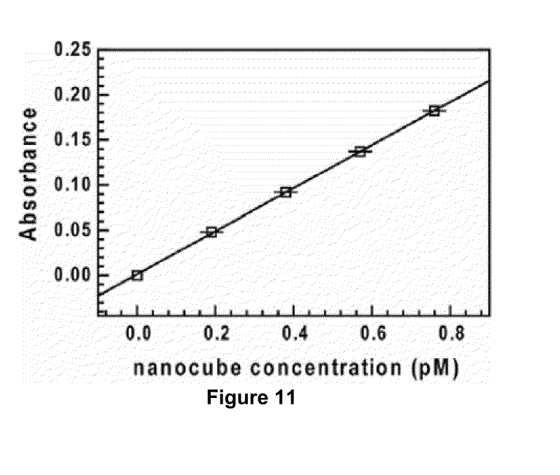

Determination of nanocube concentration in solution is necessary to evaluate the membrane surface area for kinetics calculations. To address this, nanocubes deposited onto sedimentation chambers were directly imaged by dark field scattering microscopy. A homemade image analysis program was developed to count the number of nanocubes in each imaging area. In addition, the nanocube concentration can be simply determined by measuring the absorbance using a UV-vis spectrophotometer (FIG. 11). The linear relation between particle concentration and absorbance was then used to determine the nanocube concentration during the binding measurement²⁸.

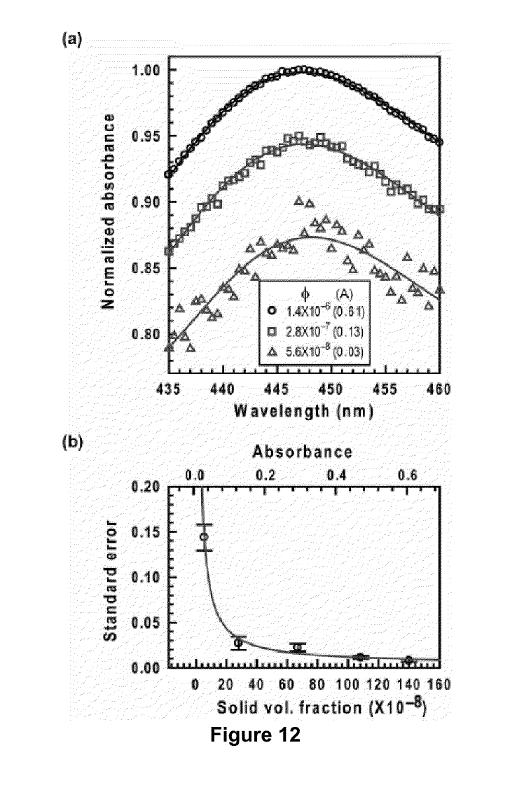

The prominent quadrupolar LSPR peak λₘₐₓ was interpolated by a polynomial fit. The higher concentration sample predictably provided a higher signal-to-noise ratio and hence higher precision of λₘₐₓ (FIG. 12a). The relation between precision of λₘₐₓ and nanocube concentration is shown in FIG. 12b. To obtain 0.01 nm precision of λₘₐₓ, working concentration of nanocube measurement is at absorbance larger than 0.4. For 10 mm optical pathlength of spectrometer cells, 0.4 absorbance corresponds to the solid volume fraction 10⁻⁶ (FIG. 12b).

This solution-based sensing platform allows the analysis of ensembles in excess of 10¹² nanocubes. In contrast to conventional LSPR assays, taking large ensemble measurements in solution reduces inaccuracies in the LSPR response caused by particle and bilayer variations thus increasing sensitivity and overall confidence in the measurement. Our calibration results provide the optimal working concentrations for the nanocube measurements. The high absorption of the nanocube sample, along with the narrow LSPR peak, results in highly precise interpolation of tiny shifts in λₘₐₓ. For the Ag@SiO₂ nanocube covered with 100% DOPC bilayer, the best resolution of LSPR measurements with a current UV-vis spectrophotometer is 0.01 nm standard error of 20 consecutive scans (standard deviation=0.04 nm). It is correspondent to a protein density change of ˜1.9·10⁻⁹ ng/μm². This indicates that the ideal sensitivity of the nanocube measurement can reach ˜22 proteins/□μm² or 1.2 proteins per nanocube for a 53 k Da size protein. The influences of protein binding may further introduce intrinsic fluctuation of signal. For example, the standard deviation of 20 measurements in Ste5 mutant system is 0.04 nm and standard error is 0.01 nm that is closed to ideal sensitivity. For Ste5 wildtype, the standard deviation and error is 0.065 nm and 0.015 nm that is a little bit higher. (FIG. 2c)

Calibration of LSPR Shifts Vs. Protein Density

To further calibrate the correlation between LSPR shift and protein surface density on the membrane, three different approaches, (1) titrating biotinyl-cap-PE in bilayer, (2) titrating streptavidins in solution, and (3) measuring unbound fluorescent streptavidins, were employed here. The first approach is to alter the mole fractions of biotinyl-cap-PE in bilayer (0%, 0.025%, 0.05%, 0.1%, and 0.2%). The bilayer coated Ag@SiO² nanocubes were incubated with excess streptavidin. By assuming a DOPC lipid footprint in supported bilayers of 0.72 nm², the average surface density of streptavidin was be calculated²⁹. This approach varies the number of biotin binding sites on the membrane surface to calibrate the dependence of the LSPR shift on protein surface density.

The second approach is to change the protein density on the membrane surface by titrating the streptavidin concentration. A fixed number of small unilamellar vesicles (SUVs, 97% DOPC+3% biotinyl-cap-PE) mixed with Ag@SiO₂ nanocubes were incubated with different amount of streptavidin. Because of the high affinity of biotin-streptavidin binding, we assume all streptavidin binds evenly and completely to vesicles and bilayers on Ag@SiO₂ nanocubes. The average streptavidin surface density on nanocubes can be evaluated by using a DOPC lipid footprint in supported bilayers.

In these two methods, we assume binding processes were complete after three hours incubation. Although previous a study shows the diffusion limitations of streptavidin binding to immobilized biotin are negligible³⁰, limited protein diffusion might erroneously lead to different calculated protein densities. Therefore, we introduce a third approach that measured unbound protein in the solution using streptavidin labeled with Alexa Fluo 647. In this approach, bilayer-coated Ag@SiO₂ nanocubes were incubated with different amount of fluorescent streptavidin for one hour. To separate bound from unbound streptavidin, streptavidin attached to bilayer-coated Ag@SiO₂ nanocubes was pulled down in a centrifuge. The concentration of unbound streptavidin remaining in the supernatant was determined by its fluorescence intensity in a spectrometer. Because nanocube concentration is known, the average streptavidin density on nanocubes was evaluated. To reduce the experimental error of fluorescent measurements, this approach required high nanocube concentrations to modulate the fluorescence intensity in supernatant.

### Direct Comparison of Multi-Component Fluorescent Correlation Spectroscopy and Nanocube Detection

Fluorescence Correlation Spectroscopy (FCS) is a quantitative tool to locally measure molecular mobility and number densities of fluorescently labeled species³¹. In multi-components FCS measurements, we first determined the average number of vesicles and CTB concentrations separately. Then, the same amount of vesicle and CTB were mixed to observe the kinetics of CTB binding. The average number of vesicles N_{T}, diffusing within the excitation spot was measured by FCS of vesicles doped with 0.5% BODIPY-FL-DHPE. These were performed with 488 nm laser excitation at 0.2 mg/ml vesicle concentration. Twenty 120 sec measurements were taken and averaged to obtain statistical variations and fitted to an analytical expression of normal 3-D diffusion in a 3D-Gaussian volume for single diffusion species:

$$G ( τ ) = \frac{1}{N} \frac{1}{1 + \frac{τ}{{τ}_{D}}} \frac{1}{\sqrt{1 + {s}^{2} \frac{τ}{{τ}_{D}}}} \qquad Eq ( 1 )$$

where N is the total number of diffusing particles, τ_{D} is the characteristic diffusion time, and s is a structure factor calibrated by a fluorescein standard. The average number of vesicles diffusing within the excitation spot Nᵥ is equal to 1/G(0) from the analytical fitting result. With the same approach, the number of Alexa 594-CTB diffusing within the excitation spot, N_{CTB}, was measured under 568 nm laser excitation. The concentration of Alex 594-CTB was 0.004 mg/ml. Finally, the same amount of Alexa 594-CTB (0.004 mg/ml) was mixed with vesicle solution (0.2 mg/ml) to reach the same concentration as the previous separate measurements. Then, the time-resolved concentration was obtained by performing a 30 sec measurement every minute using 568 nm laser excitation. For each FCS curve, the value of G(0) was extrapolated by fitting the curve to Eq. 1. Although Eq. 1 cannot fully describe multiple diffusing components with different brightnesses, it is sufficient to determine the value of G(0).

The general expression for multicomponent 3-D diffusion is:

$$G ( τ ) = \frac{1}{{( \sum {Q}_{k} {N}_{k} )}^{2}} \sum {Q}_{j}^{2} {N}_{j} \frac{1}{1 + \frac{τ}{{τ}_{Df}}} \frac{1}{\sqrt{1 + {s}^{2} \frac{τ}{{τ}_{Dj}}}} \qquad Eq ( 2 )$$

where Qk is the average brightness for the component k. In this study, we simplified the system into two components, freely diffusing and vesicle-bound Alexa 594-CTB. We assumed the average number of Alexa 594-CTB binding to one vesicle was σ. Thus, the average brightness of the CTB component on one vesicle is σ times brighter than freely diffusing Alexa 594-CTB. It has been shown that a single Q can be used to accurately represent the average properties of the true distribution in this type of measurement^{31, 32}.

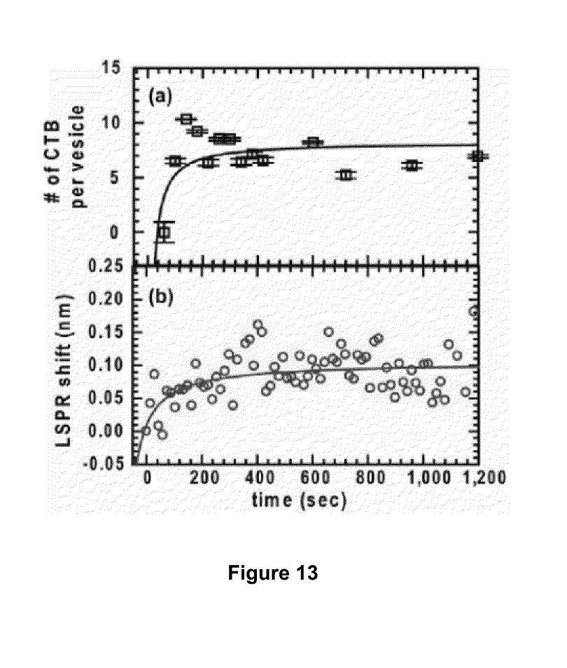

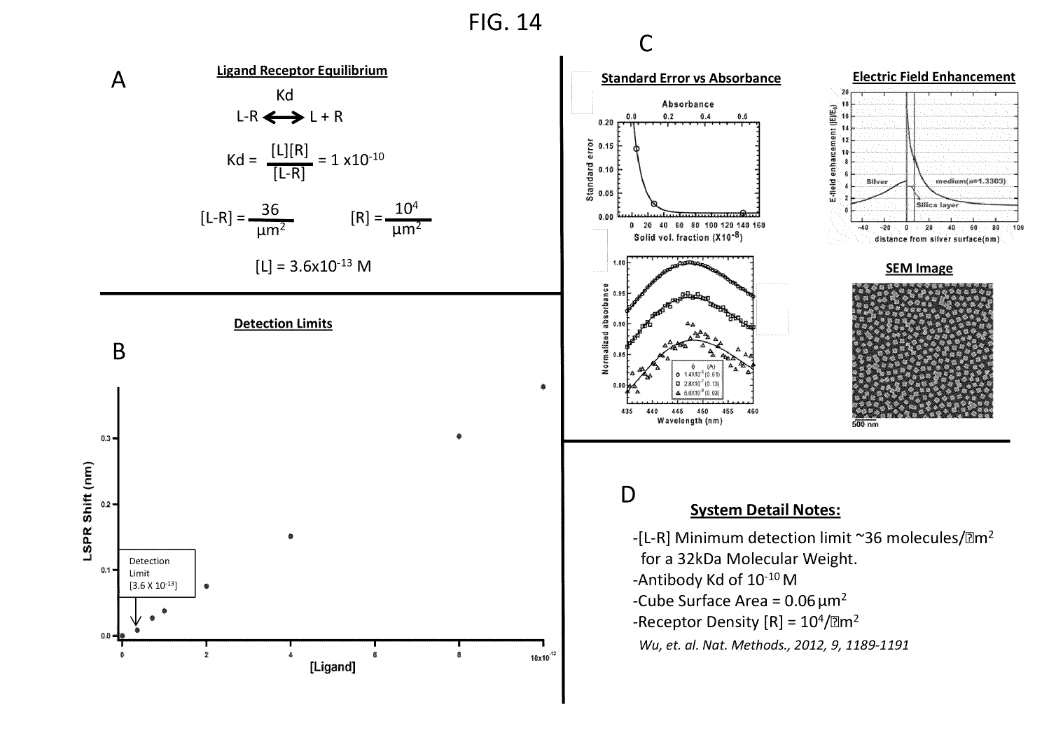

The G(0) value of equation (2) can then be expressed as

$$G ( 0 ) = \frac{{N}_{f} + {σ}^{2} {N}_{v}}{{( {N}_{f} + σ {N}_{v} )}^{2}} \qquad eq ( 3 )$$

where σ is the number of bound CTB per vesicle, and N_{f} is the number of freely diffusing Alexa 594-CTB, which can be calculated from N_{f}=N_{CTB}−σNᵥ. Using the measured Nᵥ, N_{CTB}, and G(0) values, the unknown σ can be computed from Eq (3). With the known average size of vesicles (120 nm), the surface density of CTB bound to vesicle can then be calculated (FIG. 13a).

For direct comparison, nanocube measurements were performed under the same experimental conditions as FCS. The same vesicle concentration used in FCS experiments was mixed with Ag@SiO₂ nanocubes to form supported lipid bilayers. Excess vesicles were not removed in order to maintain the same concentration of GM1 binding sites in the solution. The same amount of Alexa 594-CTB was added to the solution. Assuming that CTB binds equally to vesicles and bilayer-coated Ag@SiO₂ nanocubes, the surface density of bound CTB is the same on both surfaces. LSPR shifts were then monitored using a UV-Vis spectrophotometer (FIG. 13b). The LSPR shifts were converted to surface density using the LSPR response to protein mass change measured in the biotin-streptavidin system (0.191 ng mm⁻² nm-1, Table 1). Kinetic binding curves measured by FCS and the nanocube assay reached equilibrium after 1000 sec (FIG. 13). The suitable working range for FCS depends on the size of the detection volume and the brightness of the fluorophores, and it typically falls below 100 nM³³. Because concentration fluctuations from the ensemble average are crucial for FCS, these experiments were performed at a relatively low protein concentration and hence lower LSPR shift. Although the kinetic binding curves show a lower signal-to-noise ratio under such experimental conditions, the binding curves and final bound CBT density obtained from the two methods still show excellent agreement. In contrast to FCS, the detection of nanocube assay is not limited by analyte concentration because it measures the change of local refractive index. Practically, we have successfully performed protein binding measurement at concentration in the hundreds of micromolar range.

### Detergent Effect

During the measurement of Ste5-PH domain binding on supported phospholipid bilayers, we speculated that desorption of the lipid bilayer could influence the LSPR response. From our observations, adding detergent caused a blue shift that we attribute to disruptions of the bilayer. Detergents with low critical micelle concentration and high molecular weight are difficult to remove by either dialysis or gel filtration³⁴. Our results suggest that the use of detergent should be eliminated in all protein preparation steps for membrane protein binding measurements. In this paper, the use of detergent was therefore eliminated during protein purification to avoid these effects.

### Methods

### Materials

Lipids. The following lipids were purchased from Avanti Polar Lipids (Alabaster, AL): 1,2-dioleoyl-sn-glycero-3-phosphocholine (DOPC), 1,2-dioleoyl-sn-glycero-3-phosphoethanolamine-N-(cap-biotinyl) (Biotinyl-Cap-PE), Ganglioside GM1 (GM1), 1,2-dioleoylsn-glycero-3-phospho-L-serine (DOPS),1,2-dioleoyl-sn-glycero-3-phosphate (DOPA), 1,2-dioleoyl-sn-glycero-3-phosphoethanolamine (DOPE), L-α-phosphatidylinositol (PI), and L-α-phosphatidylinositol-4,5-bisphosphate (PI(4,5)P₂). The fluorescent lipid probes, Texas Red 1,2-dipalmitoyl-sn-glycero-3-phosphoethanolamine (Texas red DPPE) and N-(4,4-difluoro-5,7-dimethyl-4-bora-3a,4a-diaza-s-indacene-3-propionyl)-1,2-dihexadecanoyl-sn-glycero-3-phosphoethanolamine, triethylammonium salt (BODIPY-FL-DHPE), were purchased from Invitrogen.

Ethanol (200 proof), tetraethyl orthosilicate (TEOS), 28% ammonium hydroxide solution, unlabeled recombinant streptavidin, and bovine serum albumin were purchased from Sigma-Aldrich. The fluorescent proteins Alexa Fluor 647 streptavidin and cholera toxin subunit B (CTB) Alexa Fluor 594 were purchased from Invitrogen. Streptavidin and CTB binding experiments were performed in 1×PBS buffer (Mediatech). GST-Ste5 binding measurements were performed in HKME buffer (20 mM HEPES-KOH at pH=7.0, 160 mM KOAc, 1 mMn MgCl₂, 0.1 mM EGTA).

### Silica-Coated Nanocube

Ag nanocubes are synthesized using the polyol method^{12, 17, 18} capped with poly(vinylpyrrolidone) (PVP), and stored in ethylene glycol before use. Silica shells were coated on Ag nanocubes using Stöber process.¹⁹ The concentration of ammonium hydroxide and reaction time affected the thickness and quality of the silica layer.²⁰ Ag nanocubes were first washed extensively with ethanol. Silica layers were coated by mixing 7.5 ml of Ag nanocube suspension in ethanol with 1950 μl of water, 600 μl of TEOS, and 300 μl of 0.28% ammonium hydroxide. The solution was sonicated during the entire reaction. After 40 min reaction, the Ag@SiO₂ nanocubes were washed with ethanol to remove the reagents and then washed extensively with water. The Ag@SiO₂ nanocubes were stored in deionized water for future use.

### LSPR Measurement

Various approaches have been reported to collect nanoparticle extinction spectra21. We employed a general transmission ultraviolet-visible (UV-vis) spectrophotometer (Cary 100, Varian). Typically, spectral shifts were monitored by detecting the prominent quadrupolar LSPR peak λmax. These peaks were determined by fitting transmission spectra to a seventh-order polynomial (FIG. 1g). The dependence of LSPR peak shift on refractive index was measured in water-glycerol solutions of various ratios. To explore the effect of the silica shell, the refractive index sensitivity of Ag@SiO₂ nanocubes was compared to Ag nanocubes using solutions of water and glycerol. (FIG. 10) LSPR sensitivity was quantified using the widely reported figure of merit (FOM) calculated by dividing refractive index sensitivity by the line width of resonance spectrum (FOM=S/Δλ)^{22, 23} The refractive index sensitivity (S) was evaluated from FIG. 10 and represented as peak shift (reported in nm or eV) per refractive index unit (RIU). The line width of the resonance spectrum (Al) was obtained from the full width at half maximum (FWHM) of the LSPR peak (FIG. 1g).

To demonstrate the applicability of other detection schemes, scattering spectra were also measured by (1) dark field scattering microscopy using a dark field condenser and spectrometer (USB2000, Ocean Optics), and (2) a fluorescence spectrophotometer (Varian, Inc.) configured for 90 degree scattering detection. The nanocube concentrations were determined by counting deposited nanocubes on glass substrates. The silica-coated nanocube solutions were incubated in a sedimentation chamber for two days to create monolayers of nanocubes. Dark field microscopy was used to observe the nanocubes deposited on the bottom of each sedimentation chamber. A homemade image analysis program was developed to count the number of nanocubes in each imaging frame.

### Bilayer Preparation

Lipid vesicles. The desired composition of lipids was first mixed in chloroform. The mixture was then dried in a round bottom flask followed by desiccation under nitrogen for at least 30 minutes. Lipid films were then hydrated with 18.2 MΩ·cm deionized (DI) water. The resulting suspension was probe sonicated to clarity in an ice bath and ultracentrifuged at 4° C. for 45 min. The top small unilamellar vesicle (SUV) solution was extracted for use in experiments. For FCS and GST-Ste5 binding experiments, SUVs were prepared through an extrusion process. Instead of sonicating, the hydrated lipids were extruded through 100 nm polycarbonate pore filters (Whatman, UK) until the suspension reached clarity. The vesicle used in FCS measurement contains 0.5% G_{M1}, 0.5% BODIPY-FL-DHPE and 99% DOPC lipids. The lipid membranes used in GST-Ste5 PH binding experiment contain: (1) 53% DOPC, 22% DOPE, 10% DOPS, 5% DOPA, 10% PI for PIP₂-free bilayer and (2) 53% DOPC, 22% DOPE, 10% DOPS, 5% DOPA, 5% PI, 5% PI(4,5)P₂ for PIP₂ bilayer.

Supported lipid bilayers. Supported lipid bilayers were formed by adapting a standard vesicle fusion technique³. Bilayers were assembled by combining equal volumes of SUV suspension and the desired buffer in a small centrifuge tube, followed by vortex mixing. Excess vesicles and salt were removed by rinsing twice with the buffer using a benchtop centrifuge (minicentrifuge, VWR, maximum RCF=2000 g). Membrane-coated particles were then diluted to the desired working concentration and introduced into the spectrophotometer cell.

### Protein Binding Measurement

Bilayer-coated nanocubes were incubated with 0.05 mg ml⁻¹ BSA solution to block nonspecific binding prior to adding desired proteins. Fifteen consecutive scans were performed to obtain the average λₘₐₓ of the LSPR quadrupolar peak as a baseline. The desired amount of protein was directly cast into the spectrophotometer cell (400 μL sample volume) followed by pulse vortexing of the mixture. Spectra in the range of 430 nm to 480 nm were scanned immediately after mixing at 0.5 nm spectral resolution. The maximum attainable scanning rate was six seconds per spectrum, limited by the configuration of the UV-vis spectrophotometer. To minimize the use of protein in GST-Ste5 binding experiments, these measurements were performed with a sub-microvolume optical cuvette. Different volumes of protein (0.5-15 μl) were incubated with 20 μl of bilayer-coated Ag@SiO₂ nanocube sensors for two hours. The average λₘₐₓ of the LSPR quadrupolar peak were obtained from ten consecutive spectra. All experiments were performed at room temperature.

### Fluorescent Correlation Spectroscopy

Fluorescence correlation spectroscopy (FCS) measurements were performed on a homebuilt FCS apparatus based on a Nikon TE2000 inverted fluorescence microscope as described previously 24. Two laser beams, 488 nm and 568 nm, were coupled into an optical fiber and focused by a 100×TIRF objective (Nikon) onto the sample to excite the fluorescent probes. The emitted light was filtered through notch filters and a confocal pinhole then separated by a 560 nm long-pass filter. Before focusing onto two avalanche photodiodes (APDs) (Perkin and Elmer), two color filters were used to minimize spectrum crosstalk. The photon arrival time was recorded and the auto-correlation functions of the two APD signals were calculated with a hardware correlator (Correlator.com) in real time. Using a double-labeled supported lipid bilayer as a sample, overlapping detection volumes were obtained by careful alignment of a collimator lens after the optical fiber and fine adjustment of the objective lens correction collar²⁵. Measurements were made in eight-well chambered coverglass (Nunc) that were first soaked with 0.1 M NaOH for 20 min to clean the bottom surface. The supported lipid bilayers (100% DOPC) were formed on the bottom surface by vesicle fusion. The chamber was incubated with 0.1 mg/ml BSA to prevent the protein and vesicle absorption. The size and the structure factor s of the excitation volume were calibrated using 200 nM fluorescein in 1M NaOH solution with a known diffusion coefficient (D=300 μm² s⁻¹)²⁶. All other measurements were performed at 24.5° C. in 1×PBS buffer.

The model system, CTB binding to vesicles containing the membrane associated receptor monosialoganglioside GM1, was selected to directly compare FCS and nanocube measurements. To obtain a narrow size distribution of vesicles, SUVs were prepared by the standard extrusion method described above. Vesicles of 120 nm average diameter containing 0.5% GM1, 0.5% BODIPY-FL-DHPE and 99% DOPC lipids were measured by dynamic light scattering (Brookhaven Instruments Corp.). A detailed description of the multi-component FCS calculations is shown herein.

### TEM

Ag@SiO₂ nanocubes were imaged using high-resolution transmission electron microscopy (JEOL 2100-F, 200 kV). The elemental x-ray analysis maps were generated using high-angle annular dark field scanning TEM (HAADF-STEM) with an energy dispersive x-ray spectroscopy (EDS) detector. TEM images revealed nanocube a lateral dimension of 98 nm, 19 nm radius of curvature at the edges, and silica shell thickness of 3.9 nm.

### LSPR Simulation

Finite element simulations using COMSOL were used to model the LSPR of silicacoated silver nanocubes. Free tetrahedral meshing of the geometry observed in TEM was performed in COMSOL, and further refined in the vicinity of the silica shell. The final mesh contained 359,000 tetrahedral elements, and convergence of absorption spectra within 0.1% error was confirmed by comparing results from a coarser mesh.

Frequency-domain scattered electric field solutions were computed using COMSOL's RF module for a background oscillating field of arbitrary amplitude 1 V m⁻¹. Real and imaginary refractive index dispersion was interpolated from literature tables for silver and silica²⁷. The nanocube was simulated inside a sphere of diameter 400 nm, sufficiently large for all near-field effects to be negligible at the system boundary. A perfectly matched layer (PML) was additionally incorporated to cancel any reflection artifacts in the simulation. Field solutions were calculated for 50-100 different frequencies at a time.

### GST-Ste5 Protein Preparation

GST-Ste5 PH domain fusion proteins with and without R407S K411S mutations (corresponding to Ste5 residue 369-517) were constructed, expressed, and purified from Escherichia coli as described by Garrenton et al.¹⁵ The use of Tween-20 detergent was omitted during protein purification to avoid the influence of detergent on lipid bilayers. Prior to binding experiments, GST-Ste5 proteins were treated with Amicon centrifuge filters (Millipore) for further purification and buffer exchange.

### Example 2

### Detection with Antibody Conjugation to Solution-Phase Plasmonic Nanocube Sensors

Immobilized GLYMO on Oxidized Silicon Particle.

Take Ag@SiO₂ particle and coat it with epoxy group for antibody linkage. Follow the aqueous protocol published in Chem. Mater. 1997, 9, 2577-2582, hereby incorporated by reference, or as described in Example 1. GLYMO: 3-glycidoxypropyltrimethoxysilane
1. Take 500 ul of Ag@SiO₂ particle. Use centrifuge to spin down the particles and remove most of ethanol.
2. Prepare coating solution. Prepare 50% of ethanol-water as solvent. Add GLYMO in the solvent to reach 30% wt.
3. Use 6M HCl to adjust the pH below 4.
4. Mix with particle overnight.
5. Wash particle with ethanol and follow with acetone. If the particles are not used right away, store in acetone

Coating Antibody (Example: Hepcidin Antibody) on Epoxy Group Coated Ag@SiO₂.

Coupling buffer: 1×PBS, pH=8.5; Washing buffer. 1×PBS, pH=7.4
1. Remove the acetone and wash particle with coupling buffer once. Re-disperse particles into coupling buffer. (200 ul)
2. Add 20 ul of polyclone hepcidin antibody (0.5 mg/ml). Incubate at room temperature for 2 hrs.
3. Move the tube to the cold room and incubate over night.
4. Wash particle with 1×TBS once. And incubate in TBS solution for 1 hr.
5. Wash particle with 1×PBS+0.1% BSA solution 4 times.
6. Disperse particle in 500 ul 1×PBS. Store at 4 C.

Detection Protocol.

For example, to detect the hepcidin level in human body fluids.
1. Take the desired biological fluids, including serum, plasma, urine, etc. Measure its absorption spectra as background using UV-vis spectrometer.
2. Add desired amount of antibody coated Ag@SiO₂ nanocube particles directly into the biological fluids. Measure the spectra shift using UV-vis spectrometer.
3. For kinetics study, measure the time-lapse spectra to monitor the binding kinetics.
4. For diagnostic study, incubate the antibody coated nanocube sensor with biological fluids for few hours, and then detect the shift of final spectra. The shift of spectra reflect to the level of antigen (hepcidin) in human body fluids.

### Example 3

### Silver Nanocube Functionalization with Antibody

Modification of silica coated silver nanocubes with aldehyde functional groups and capture antibodies can be carried out by the following:

Take LSPR Spectrum of untreated silica coated cubes to determine maximum wavelength of absorbance:
(a) Prepare 11-(triethoxy silyl) undecanal solution which is 1% (v/v) in a 95% Ethanol: 5% H2O solution.
(b) Add 50 uL silica coated silver nanocubes to 950 uL of 11-(triethoxy silyl) undecanal solution.
(c) Incubate at room temperature for 40 minutes with constant mixing. The timing of this reaction can be adjusted to either increase or decrease the amount of 11-(triethoxy silyl) undecanal bound to the surface of the nanocubes.
(d) Using the initial maximum LSPR absorbance before treatment with 11-(triethoxy silyl) undecanal, the LSPR shift can be monitored after reaction to determine the mass of 11-(triethoxy silyl) undecanal coupled to the silica coated silver nanocubes.
(e) Centrifuge solution at 2000 G's for 3 min, removing supernatant and rinse with nanopure water. Repeat this step 4 times to remove the excess 11-(triethoxy silyl) undecanal.
(f) Suspend silver 11-(triethoxy silyl) undecanal silane solution nanocubes into 500 uL of 0.01M PBS pH 7.0 buffer.
(g) Add 10 uL of (3 mg/mL) Antibody solution. Antibodies contain a number of primary amine groups predominately due to the presence of Lysine amino acid groups in the antibody amino acid sequence. Primary amines and aldehyde groups are reactive to form a imines or Schiff bases. The 11-(triethoxy silyl) undecanal contains an aldehyde group which will be available for conjugation to the primary amines present on the antibody again predominately via lysine amino acid residues.
(h) Incubate nanocubes with antibodies for 2 hours at room temperature with constant mixing on a rotating mixer.
(i) Monitoring LSPR Shift from its maximum prior to antibody coupling to its new maximum following the coupling procedure can determine mass of antibody coupled to the nanocubes.
(j) Add a monofunctional Polyethylene Glycol derivative of Amino Polyethylene Glycol (Molecular Weight 2000) to the nanocube solution and react for an additional 1 hour with constant mixing on a rotating mixer.
(k) Centrifuge at 2000 G's for 3 min.
(l) Remove supernatant and suspend particles in nanopure water. Repeat this step 4 times to ensure removal of excess Polyethylene glycol as well as antibodies.
(m) Suspend antibody coupled nanocubes in 0.1M PBS pH 7.0 Buffer

Take LSPR Spectrum of treated silica coated cubes to determine absorbance shift after treatment.
(a) UV-Visible spectrophotometer should be turned on 30 minutes prior to experiments to allow for warming up of the lamp filament.
(b) Appropriate blanks of nanopure water should be performed prior to experiments.
(c) A dilution of the functionalized nanocubes in 0.1M PBS buffer (pH 7.0) should be prepared in a Quartz UV-Visible cuvette to obtain a UV-Visible absorbance of greater than 0.2abs units, but less than 1.0abs units, at the maximum LSPR absorbance wavelength.
(d) An aliquot of the appropriate antigen solution can now be cast into cuvette while monitoring the LSPR maximum absorbance shift from the initial LSPR maximum absorbance as determined previously.

### REFERENCES CITED

1. Kuriyan, J. & Groves, J. T. Nat. Struct. Mol. Biol. 17, 659-665 (2010).
2. Baksh, M. M., Kussrow, A. K., Mileni, M., Finn, M. G. & Bornhop, D. J. Nat. Biotechnol. 29, 357-U173 (2011).
3. Baksh, M. M., Jaros, M. & Groves, J. T. Nature 427, 139-141 (2004).
4. Zheng, G. F., Patolsky, F., Cui, Y., Wang, W. U. & Lieber, C. M. Nat. Biotechnol. 23, 1294-1301 (2005).
5. Braun, T. et al. Nat. Nanotechnol. 4, 179-185 (2009).
6. Cooper, M. A. J. Mol. Recognit. 17, 286-315 (2004).
7. Beseni\check{c}ar, M., Ma\check{c}ek, P., Lakey, J. H. & Anderluh, G. Chem. Phys. Lipids 141, 169-178 (2006).
8. Dahlin, A. et al. J. Am. Chem. Soc. 127, 5043-5048 (2005).
9. Galush, W. J. et al. Nano Lett. 9, 2077-2082 (2009).
10. Jonsson, M. P., Jonsson, P., Dahlin, A. B. & Hook, F. Nano Lett. 7, 3462-3468 (2007).
11. Baciu, C. L., Becker, J., Janshoff, A. & Sonnichsen, C. Nano Lett. 8, 1724-1728 (2008).
12. Tao, A., Sinsermsuksakul, P. & Yang, P. D. Angew. Chem. Int. Edit. 45, 4597-4601 (2006).
13. Roiter, Y. et al. Langmuir 25, 6287-6299 (2009).
14. Middleton, E. R. & Rhoades, E. Biophys. J. 99, 2279-2288 (2010).
15. Garrenton, L. S., Young, S. L. & Thorner, J. Gene. Dev. 20, 1946-1958 (2006).
16. Zhao, C., Du, G. W., Skowronek, K., Frohman, M. A. & Bar-Sagi, D. Nat. Cell. Biol. 9, 706-U171 (2007).
17. Fievet, F., Lagier, J. P., Blin, B., Beaudoin, B. & Figlarz, M. Solid State Ionics 32-3, 198-205 (1989).
18. Sun, Y. G. & Xia, Y. N. Science 298, 2176-2179 (2002).
19. Stober, W., Fink, A. & Bohn, E. J. Colloid Interface Sci. 26, 62-& (1968).
20. Sioss, J. A., Stoermer, R. L., Sha, M. Y. & Keating, C. D. Langmuir 23, 11334-11341 (2007).
21. Willets, K. A. & Van Duyne, R. P. Annu. Rev. Phys. Chem. 58, 267-297 (2007).
22. Sherry, L. J. et al. Nano Lett. 5, 2034-2038 (2005).
23. Mayer, K. M. & Hafner, J. H. Chem. Rev. 111, 3828-3857 (2011).
24. Forstner, M. B., Yee, C. K., Parikh, A. N. & Groves, J. T. J. Am. Chem. Soc. 128, 15221-15227 (2006).
25. Bacia, K. & Schwille, P. Nat. Protoc. 2, 2842-2856 (2007).
26. Chen, Y., Muller, J. D., Eid, J. S. & Gratton, E. in In New Trends in Fluorescence Spectroscopy: Applications to Chemical and Life Sciences. (eds. Valeur, B. & Brochon, J. C.) 277-302 (Springer, Berlin, 2001).
27. Palik, E. D. Handbook of Optical Constants of Solids. (Elsevier, Amsterdam, 1998).
28. Haiss, W., Thanh, N. T. K., Aveyard, J. & Fernig, D. G. Anal. Chem. 79, 4215-4221 (2007).
29. Vacklin, H. P., Tiberg, F. & Thomas, R. K. Biochim. Biophys. Acta, Biomembr. 1668, 17-24 (2005).
30. Balgi, G., Leckband, D. E. & Nitsche, J. M. Biophys. J. 68, 2251-2260 (1995).
31. Middleton, E. R. & Rhoades, E. Biophys. J. 99, 2279-2288 (2010).
32. Pu, M. M., Fang, X. M., Redfield, A. G., Gershenson, A. & Roberts, M. F. J. Biol. Chem. 284, 16099-16107 (2009).
33. Krichevsky, O. & Bonnet, G. Rep. Prog. Phys. 65, 251-297 (2002).
34. Rigaud, J. L., Levy, D., Mosser, G. & Lambert, O. Eur. Biophys. J. 27, 305-319 (1998).
35. Siekkinen, A R, McLellan J M, Chen, J., Xia, Y, “Rapid synthesis of small silver nanocubes by mediating polyol reduction with a trace amount of sodium sulfide or sodium hydrosulfide,”Chem Phys Lett. 2006 Dec. 11; 432(4-6): 491-496

The references cited above are hereby incorporated by reference.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A polyhedral nanoparticle having a core coated with a continuous layer of silica, further coated with a continuous membrane and having a ligand attached to said nanoparticle.

2. The nanoparticle of claim 1, wherein the membrane is a lipid bilayer.

3. The nanoparticle of claim 1, wherein the nanoparticle is a nanocube.

4. The nanoparticle of claim 3, wherein the nanocube is 100 to 500 nm in edge length.

5. The nanoparticle of claim 3, wherein the nanoparticle core comprises a metal, a semiconductor material, multi-layers of metals, a metal oxide, an alloy, a polymer, or a carbon nanomaterial.

6. The nanoparticle of claim 1, wherein the nanoparticle core comprises gold or silver.

7. The nanoparticle of claim 1, wherein the ligand is deposed with the membrane, wherein the ligand is capable of binding to an analyte molecule.

8. The nanoparticle of claim 7, wherein the ligand is an antibody, a protein, a functionalized lipid headgroup, a drug, a nucleic acid, an oligonucleotide, a peptide, or a small molecule.

9. The nanoparticle of claim 7, wherein the analyte molecule is a cell-surface protein, a functionalized lipid headgroup, an antibody binding pair, drugs, metabolites, pesticides, pollutants, bacteria, or a cell bearing a blood group antigen, an HLA antigen or a microorganism, bacterium, fungus, protozoan, or virus.

10. The nanoparticle of claim 1, wherein the ligand is attached to the nanoparticle by a linker from the group consisting of silane, thiol or epoxy.

11. A sensing device comprising a nanoparticle of claim 1 deposed on a substrate.

12. A method comprising: (a) providing a sensing device of claim 11, the method comprising the steps: (a) providing a solution, wherein the solution is suspected of containing a target molecule, (b) contacting the solution with a ligand conjugated to a nanoparticle of the invention and allowing the ligand conjugated to the nanoparticle to bind any target molecule present in the solution, and (c) detecting plasmon generated phenomena at the nanoparticle by the binding of the target molecule to the ligand conjugated to the nanoparticle

13. The method of claim 12, wherein the detecting step comprises detecting a optical detectable change.

14. The method of claim 13, wherein the detecting step comprises detecting a spectral shift in the known spectra of the nanoparticle, wherein the spectral shift indicates the presence of a molecule possibly capable of binding the target molecule.

15. A method for detecting an analyte of interest comprising: (a) providing a polyhedral nanoparticle having a core coated with a continuous layer of silica and having a ligand attached to said nanoparticle, wherein the nanoparticle has a known spectra, and wherein a membrane of the nanoparticle displays a ligand for the analyte of interest; (b) applying a sample suspected of containing a target analyte of interest to the nanoparticle; (c) detecting plasmon generated phenomena at the nanoparticle, whereby a spectral shift in the known spectra of the nanoparticle indicates that the target analyte is bound to the ligand.

16. The nanoparticle of claim 15, wherein the nanoparticle is a nanocube, wherein the nanocube is 100 to 500 nm in edge length.

17. The nanoparticle of claim 16, wherein the nanoparticle core comprises gold or silver.

18. The nanoparticle of claim 17, wherein the nanoparticle core is silver and 100 nm in edge length.

19. The nanoparticle of claim 17, wherein the ligand is an antibody, a protein, a functionalized lipid headgroup, a drug, a nucleic acid, an oligonucleotide, a peptide, or a small molecule.

20. The nanoparticle of claim 17, wherein the analyte molecule is a cell-surface protein, a functionalized lipid headgroup, an antibody binding pair, drugs, metabolites, pesticides, pollutants, bacteria, or a cell bearing a blood group antigen, an HLA antigen or a microorganism, bacterium, fungus, protozoan, or virus.

21. The nanoparticle of claim 17, wherein the ligand is attached to the nanoparticle by a linker from the group consisting of silane, thiol or epoxy.
